(19) United States
(12) Patent Application Publication
(10) Pub. No.: US 2010/0279281 A1
(43) Pub. Date: Nov. 4, 2010

(54) METHOD OF IDENTIFYING LUNG CANCERS ASSOCIATED WITH ASBESTOS-EXPOSURE

(75) Inventors: Sisko Anttila, Espoo (FI); Sakari Knuutila, Espoo (FI); Jaakko Hollmen, Espoo (FI); Salla Ruosaari, Espoo (FI); Harriet Wikman-Kocher, Hamburg (DK); Penny Nymark, Helsinki (FI)

Correspondence Address: BIRCH STEWART KOLASCH & BIRCH, PO BOX 747, FALLS CHURCH, VA, 22040-0747, US

(73) Assignee: LICENTIA LTD., Helsinki (FI)

(21) Appl. No.: 12/161,003

(22) PCT Filed: Jan. 18, 2007
(86) PCT No.: PCT/FI2007/050023

§ 371 Date: Oct. 15, 2008

(30) Foreign Application Priority Data
Jan. 18, 2006 (FI) 20065032

(51) Int. Cl. C12Q 1/68; G01N 33/50

(52) U.S. Cl. 435/6; 436/94

(57) ABSTRACT

The present invention is related to a method for assessing the presence of, or disposition to, an asbestos-related disorder in a subject. Particularly, the invention provides a method of identifying lung cancers associated with asbestos-exposure. The association is confirmed by the detection of allelic imbalance (AI) in at least one of the following chromosomal regions of lung cancer cells: 19p13.3-p13.1; 9q32-34.3; 2p21-p16.3; 16p13.3; 22q12.3-q13.1; and 5q35.3.

### FIELD OF THE INVENTION

The present invention is based on a molecular level description of genomic alterations in lung cancer cells. The invention provides a method of identifying lung cancers associated with asbestos-exposure by detecting allelic imbalance (AI) in DNA derived from lung cancer cells. Also, the present invention shows that asbestos exposed lung cancer patients have a distinct gene expression profile in their lung carcinomas. The invention also provides a method that may be used for early detection, prediction, and prevention of asbestos-related lung cancer by detection of AI, or RNA or protein alterations resulting from asbestos-related genomic changes, in body fluids of asbestos-exposed individuals.

### BACKGROUND OF THE INVENTION

Lung cancer is the leading cause of cancer with more than 1 million deaths a year. Tobacco smoking is undoubtedly the single most important reason of lung cancer. In addition to tobacco, lung cancer is associated with occupational and environmental exposure to other carcinogenic factors such as asbestos. Tobacco smoking together with asbestos-exposure have been shown to act synergistically leading to more than an additive effect on the risk of lung cancer (Selikoff, 1968; Vainio, 1994). The etiologic fraction of asbestos exposure in lung cancer among men has been estimated to range between 6% and 23% in different populations (Karjalainen, 1997; Nelson, 2002).

Asbestos is a group of fibrous silicate minerals that are classified into six types based on different chemical and physical features. Their insulating, fireproofing, and reinforcement properties have made them widely exploited in industry. Owing to the long latency period between the initial exposure to asbestos and disease, which has been estimated to take longer than 20 years from the onset of exposure, asbestos will keep causing disease also in countries, where the use of asbestos has been banned (for review see Consensus report in Scandinavian Journal of Work, Environment, and Health, 1997, 23:311-316).

Asbestos has been shown to be a genotoxic and cytotoxic agent that can produce both DNA and chromosomal damage. The mechanisms behind these actions may be multiple. The main mechanisms are thought to be generation of reactive oxygen (ROS) and nitrogen species (RNS), physical disturbance of cell cycle progression, and activation of several signal transduction pathways (Upadhyay, 2003; Jaurand, 1997).

Asbestos-exposed workers have been reported to have increased levels of sister chromatid exchanges and DNA double-strand breaks in white blood cells (Fatma, 1991; Marczynski, 1994). Elevated concentrations of 8-hydroxy-2′-deoxyguanosine (8-OHdG) DNA adducts, a marker for ROS exposure, have been detected both in the blood and lung tissue of asbestos-exposed workers (Marczynski, 2000). Moreover, Marsit et al. (2004) disclose that loss of heterozygosity of chromosome 3p21 in lung cancer cells is associated with occupational asbestos exposure.

Today, the clinical diagnosis of asbestos-related diseases, such as asbestos-related lung cancer, is based on a detailed interview of the patient and occupational data on asbestos exposure, appropriate latency and symptoms, and radiological and lung physiology findings (see Consensus report, 1997). However, clinical signs and symptoms of asbestos-related lung cancer do not differ from those of lung cancer of other causes. Thus, the problem of the art is that because of the high incidence of lung cancer in the general population, it is not possible to prove in precise terms that asbestos is the causative for a lung cancer in an individual patient, even when asbestosis is present. The solution provided by the present invention is the discovery of five distinct chromosomal regions that are prone to allelic imbalance in asbestos-related lung cancers. Thus, the present invention is able to provide a method for identifying asbestos-related lung cancers from the other lung cancers by detecting the presence or absence of allelic imbalance in the certain parts of the chromosome of lung cancer cells.

### SUMMARY OF THE INVENTION

The present invention provides a method and a kit for identifying lung cancers associated with asbestos-exposure, the method comprising steps of providing a sample of lung cancer cells taken from an individual suffering from lung cancer or at risk of lung cancer due to asbestos-exposure and detecting the type of allelic imbalance (AI) characteristic to asbestos-associated lung cancer in at least one of the following chromosomal regions of said lung cancer cells:

a) 19p13.3-p12; b) 9q32-34.3; c) 2p21-p16.3; d) 16p13.3; e) 22q12.3-q13.1; and f) 5q35.3. The asbestos-associated AI found in these chromosomal regions may extend beyond these regions.

### DETAILED DESCRIPTION OF THE INVENTION

### I. Definitions

The term “asbestosis” is defined as diffuse interstitial fibrosis of the lung as a consequence of exposure to asbestos dust.

The term “allelic imbalance” (AI) is defined as a situation where one member (i.e. an allele) of a gene pair is lost (i.e. a loss of heterozygosity, LOH) or amplified. Allelic imbalance thus refers to a situation where a copy number of one of the alleles is altered in a chromosome.

The terms “nucleic acid,” “polynucleotide” and “oligonucleotide” are used interchangeably and refer to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof.

The term “target nucleic acid” refers to a nucleic acid (often derived from a biological sample), to which a polynucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid can refer to the specific subsequence of a larger nucleic acid to which the probe is directed or to the overall sequence (e.g., gene or mRNA) whose expression level it is desired to detect.

A “probe” or “polynucleotide probe” is an nucleic acid capable of binding to a target nucleic acid of complementary sequence through one or more types of chemical bonds, usually through complementary base pairing, usually through hydrogen bond formation, thus forming a duplex structure. The probe binds or hybridizes to a “probe binding site.” A probe can include natural (i.e., A, G, C, or T) or modified bases (7-deazaguanosine, inosine, etc.). A probe can be an oligonucleotide which is a single-stranded DNA. Polynucleotide probes can be synthesized or produced from naturally occurring polynucleotides. In addition, the bases in a probe can be joined by a linkage other than a phosphodiester bond, so long as it does not interfere with hybridization. Thus, probes can include, for example, peptide nucleic acids in which the constituent bases are joined by peptide bonds rather than phosphodiester linkages (see, e.g., Nielsen et al., Science 254, 1497-1500 (1991)). Some probes can have leading and/or trailing sequences of noncomplementarity flanking a region of complementarity. A “perfectly matched probe” has a sequence perfectly complementary to a particular target sequence. The probe is typically perfectly complementary to a portion (subsequence) of a target sequence. The term “mismatch probe” refer to probes whose sequence is deliberately selected not to be perfectly complementary to a particular target sequence.

A “primer” is a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides, although shorter or longer primers can be used as well, Short primer molecules generally require cooler temperatures to form sufficiently stable hybrid complexes with the template. A primer need not reflect the exact sequence of the template but must be sufficiently complementary to hybridize with a template. The term “primer site” refers to the area of the target DNA to which a primer hybridizes. The term “primer pair” means a set of primers including a 5′ “upstream primer” that hybridizes with the 5′ end of the DNA sequence to be amplified and a 3′ “downstream primer” that hybridizes with the complement of the 3′ end of the sequence to be amplified.

The term “complementary” means that one nucleic acid is identical to, or hybridizes selectively to, another nucleic acid molecule. Selectivity of hybridization exists when hybridization occurs that is more selective than total lack of specificity. Typically, selective hybridization will occur when there is at least about 55% identity over a stretch of at least 14-25 nucleotides, preferably at least 65%, more preferably at least 75%, and most preferably at least 90%. Preferably, one nucleic acid hybridizes specifically to the other nucleic acid. See M. Kanehisa, Nucleic Acids Res. 12:203 (1984).

The terms “polypeptide,” “peptide” and “protein” are used interchangeably to refer to a polymer of amino acid residues. The term also applies to amino acid polymers in which one or more amino acids are chemical analogues of a corresponding naturally occurring amino acids.

The terms “identical” or percent “identity,” in the context of two or more nucleic acids or polypeptides, refer to two or more sequences or subsequences that are the same or have a specified percentage of nucleotides or amino acid residues that are the same, when compared and aligned for maximum correspondence, as measured using a sequence comparison algorithm such as those described below for example, or by visual inspection.

The phrase “substantially identical,” in the context of two nucleic acids or polypeptides, refers to two or more sequences or subsequences that have at least 75%, preferably at least 85%, more preferably at least 90%, 95% or higher nucleotide or amino acid residue identity, when compared and aligned for maximum correspondence, as measured using a sequence comparison algorithm such as those described below for example, or by visual inspection. Preferably, the substantial identity exists over a region of the sequences that is at least about 30 residues in length, preferably over a longer region than 50 residues, more preferably at least about 70 residues, and most preferably the sequences are substantially identical over the full length of the sequences being compared, such as the coding region of a nucleotide for example. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, e.g., Current Protocols in Molecular Biology (Ausubel et al., 1995 supplement).

One useful algorithm for conducting sequence comparisons is PILEUP. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity is the BLAST and the BLAST 2.0 algorithms, which are described in Altschul et al., J. Mol. Biol. 215:403-410 (1990). Software for performing BLAST analyses is publicly available through the National Center for Biotechnology Information (http://www.ncbi.nlm.nih govl). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra.). These initial neighborhood word bits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM 62 scoring matrix. The TBLATN program (using protein sequence for nucleotide sequence) uses as defaults a word length (W) of 3, an expectation (E) of 10, and a BLOSUM 62 scoring matrix. (See, e.g., Henikoff & Henikoff, Proc. Natl. Acad. Sci. USA 89:10915 (1989)).

Another indication that two nucleic acid sequences are substantially identical is that the two molecules hybridize to each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide.

A further indication that two nucleic acid sequences or polypeptides are substantially identical is that the polypeptide encoded by the first nucleic acid is immunologically cross reactive with the polypeptide encoded by the second nucleic acid, as described below. The phrases “specifically binds to a protein” or “specifically immunoreactive with,” when referring to an antibody refers to a binding reaction which is determinative of the presence of the protein in the presence of a heterogeneous population of proteins and other biologics. Thus, under designated immunoassay conditions, a specified antibody binds preferentially to a particular protein and does not bind in a significant amount to other proteins present in the sample. Specific binding to a protein under such conditions requires an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

A polypeptide is typically substantially identical to a second polypeptide, for example, where the two peptides differ only by conservative substitutions. A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well-known in the art. See, e.g., Creighton (1984) Proteins, W.H. Freeman and Company. In addition, individual substitutions, deletions or additions which alter, add or delete a single amino acid or a small percentage of amino acids in an encoded sequence are also “conservatively modified variations.”

The term “naturally occurring” as applied to an object refers to the fact that an object can be found in nature. For example, a polypeptide or polynucleotide sequence that is present in an organism that can be isolated from a source in nature and which has not been intentionally modified by humans in the laboratory is naturally occurring.

The term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

### II. Overview

Many biological functions are controlled through changes in the expression of various genes by transcriptional (e.g., through control of initiation, RNA processing, etc.) and/or translational control. For example, fundamental biological processes such as cell cycle, cell differentiation and cell death, are often characterized by the variations in the expression levels of groups of genes (see e.g. WO02059271). The changes in gene expression also are associated with pathogenesis. Thus, changes in the expression levels of particular genes can indicate the presence and progression of various diseases.

According to the invention, genes that are differentially expressed in asbestos-related lung cancer have been discovered. One or more of these target genes can be used as part of an “an expression profile” that is representative of a particular state of a lung cancer. Identification of these new target genes enable lung cancers to be analyzed more reliably. These results also provide new insights into carcinogenic mechanisms related to asbestos exposure and reveal new potential target genes for the therapy of asbestos-related diseases such as lung cancers. These differentially expressed genes and their corresponding proteins can also be utilized as “markers” that characterize particular cellular states for lung cancer cells.

The differentially expressed genes that have been identified can be utilized in a variety of methods for classifying lung cancers, as well as diagnosing and treating other asbestos-mediated diseases (e.g., asbestosis, pleural disorders, and mesothelioma). Kits and devices including one or more of the differentially expressed genes, proteins encoded by these genes and/or antibodies, primers and probes that bind the proteins or the genes are also provided.

For example, the differentially expressed genes can be used to in screening methods to identify compounds that modulate the expression or activity of the differentially expressed genes. Such methods can be utilized, for example, for the identification of compounds that can treat symptoms of disorders related to expression of proteins encoded by the differentially expressed genes. In addition, the invention encompasses methods for treating lung cancers by administering compounds and/or other substances that modulate the activity of one or more of the target genes or target gene products. Such compounds and other substances can effect the modulation either on the level of target gene expression or target protein activity. Certain classification methods that are also provided involve determining the level of one or more of the differentially expressed genes to determine whether a lung cancer is caused by asbestos exposure or not. Differentially expressed genes may also be used to develop methods for early diagnosis, prediction, and prevention of lung cancer in individuals at risk of lung cancer due to their exposure to asbestos.

### III. Differentially Expressed Genes

As described more fully in the examples below, an initial set of experiments were conducted to identify the gene expression profiles of lung cancer cells. This allowed those genes involved in asbestos exposure to be identified. The differentially expressed genes include, for instance, those listed in Table 5.

As discussed in greater detail below, knowledge of the nucleic acids that are up-regulated or down-regulated in the various types of lung cancers provides the basis for a number of different screening, treatment and diagnostic methods, in addition to devices to carry out these methods. Expression profiles as used herein refers to the pattern of gene expression corresponding to at least one differentially expressed genes, but typically includes a plurality of genes. For instance, an expression profile can include at least 1, 2, 3, 4 or 5 differentially expressed genes, but in other instances can include at least 6, 7, 8, 9, 10, 12, 14, 16, 18, 20, 25, 30, 35, 40, 45 or 50 or more differentially expressed genes. In some instances, expression profiles include all of the differentially expressed genes known for a particular type of lung cancer cell. So, for example, certain expression profiles include a measure (quantitative or qualitative) of the expression level for each of the differentially expressed genes in Table 5.

The pattern of expression associated with gene expression profiles can be defined in several ways. For example, a gene expression profile can be the absolute (e.g., a measured value) or relative transcript level of any number of particular differentially expressed genes. In other instances, a gene expression profile can be defined by comparing the level of expression of a variety of genes in one state to the level of expression of the same genes in another state (e.g., activated versus unactivated), or between one cell type and another cell type.

As used herein, the term “differentially expressed gene” or “differentially expressed nucleic acid” refers to the specific sequence as set forth in the particular GenBank entry that is provided herein (see, e.g., the Tables). The term, however, is also intended to include more broadly naturally occurring sequences (including allelic variants of those listed for the GenBank entries), as well as synthetic and intentionally manipulated sequences (e.g., nucleic acids subjected to site-directed mutagenesis). It is noted that the sequences of the target genes listed in the tables are available in the public databases. The tables provide the accession number and name for each of the sequences. The sequences of the genes in GenBank are herein expressly incorporated by reference in their entirety as of the filing date of this application (see www.ncbi.nim.nih.gov).

Differentially expressed nucleic acids also include sequences that are complementary to the listed sequences, as well as degenerate sequences resulting from the degeneracy of the genetic code. Thus, the differentially expressed nucleic acids include: (a) nucleic acids having sequences corresponding to the sequences as provided in the listed GenBank accession number; (b) nucleic acids that encode amino acids encoded by the nucleic acids of (a); (c) a nucleic acid that hybridizes under stringent conditions to a complement of the nucleic acid of (a); and (d) nucleic acids that hybridize under stringent conditions to, and therefore are complements of, the nucleic acids described in (a) through (c). The differentially expressed nucleic acids of the invention also include: (a) a deoxyribonucleotide sequence complementary to the full-length nucleotide sequences corresponding to the listed GenBank accession numbers; (b) a ribonucleotide sequence complementary to the full-length sequence corresponding to the listed GenBank accession numbers; and (c) a nucleotide sequence complementary to the deoxyribonucleotide sequence of (a) and the ribonucleotide sequence of (b). The differentially expressed nucleic acids further include fragments of the foregoing sequences. For example, nucleic acids including 10, 12, 14, 16, 18, 20, 22, 24, 26, 28, 30, 40, 50, 60, 70, 80, 90, 100, 125, 150, 175, 200, 225, 250, 275 or 300 contiguous nucleotides (or any number of nucleotides therebetween) from a differentially expressed nucleic acid are included. Such fragments are useful, for example, as primers and probes for hybridizing full-length differentially expressed nucleic acids (e.g., in detecting and amplifying such sequences).

In some instances, the differentially expressed nucleic acids include conservatively modified variations. Thus, for example, in some instances, the differentially expressed nucleic acids are modified. One of skill will recognize many ways of generating alterations in a given nucleic acid construct. Such well-known methods include site-directed mutagenesis, PCR amplification using degenerate polynucleotides, exposure of cells containing the nucleic acid to mutagenic agents or radiation and chemical synthesis of a desired polynucleotide (e.g., in conjunction with ligation and/or cloning to generate large nucleic acids). See, e.g., Giliman and Smith (1979) Gene 8:81-97, Roberts et al. (1987) Nature 328: 731-734). When the differentially expressed nucleic acids are incorporated into vectors, the nucleic acids can be combined with other sequences including, but not limited to, promoters, polyadenylation signals, restriction enzyme sites and multiple cloning sites. Thus, the overall length of the nucleic acid can vary considerably.

As described above, sequence identity comparisons can be conducted using a nucleotide sequence comparison algorithm such as those know to those of skill in the art. For example, one can use the BLASTN algorithm. Suitable parameters for use in BLASTN are wordlength (W) of 11, M=5 and N=−4 and the identity values and region sizes just described.

### IV. Preparation of Differentially Expressed Genes

The differentially expressed nucleic acids can be obtained by any suitable method known in the art, including, for example: (1) hybridization of genomic or cDNA libraries with probes to detect homologous nucleotide sequences; (2) antibody screening of expression libraries to detect cloned DNA fragments with shared structural features; (3) various amplification procedures such as polymerase chain reaction (PCR) using primers capable of annealing to the nucleic acid of interest; and (4) direct chemical synthesis.

The desired nucleic acids can also be cloned using well-known amplification techniques. Examples of protocols sufficient to direct persons of skill through in vitro amplification methods, including the polymerase chain reaction (PCR) the ligase chain reaction (LCR), Qβ-replicase amplification and other RNA polymerase mediated techniques, are found in Berger, Sambrook, and Ausubel, as well as Mullis et al. (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039.

As an alternative to cloning a nucleic acid, a suitable nucleic acid can be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method described in U.S. Pat. No. 4,458,066. Chemical synthesis produces a single stranded polynucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences can be cloned and the appropriate subsequences cleaved using appropriate restriction enzymes. The fragments can then be ligated to produce the desired DNA sequence.

### V. Utility of Differentially Expressed Nucleic Acids and Expression Profiles

As alluded to above and described in greater detail below, the differentially expressed nucleic acids that are provided can be used as markers in a variety of screening and diagnostic methods. For example, the differentially expressed nucleic acids find utility as hybridization probes or amplification primers. In certain instances, these probes and primers are fragments of the differentially expressed nucleic acids of the lengths described earlier in this section. Such fragments are generally of sufficient length to specifically hybridize to an RNA or DNA in a sample obtained from a subject. The nucleic acids are typically 10-30 nucleotides in length, although they can be longer as described above. The probes can be used in a variety of different types of hybridization experiments, including, but not limited to, Northern blots and Southern blots and in the preparation of custom arrays (see infra). The differentially expressed nucleic acids can also be used in the design of primers for amplifying the differentially expressed nucleic acids and in the design of primers and probes for quantitative RT-PCR. The primers most frequently include about 20 to 30 contiguous nucleotides of the differentially expressed nucleic acids to obtain the desired level of stability and thus selectivity in amplification, although longer sequences as described above can also be utilized.

Hybridization conditions are varied according to the particular application. For applications requiring high selectivity (e.g., amplification of a particular sequence), relatively stringent conditions are utilized, such as 0.02 M to about 0.10 M NaCl at temperatures of about 50° C. to about 70° C. High stringency conditions such as these tolerate little, if any, mismatch between the probe and the template or target strand of the differentially expressed nucleic acid. Such conditions are useful for isolating specific genes or detecting particular mRNA transcripts, for example.

Other applications, such as substitution of amino acids by site-directed mutagenesis, require less stringency. Under these conditions, hybridization can occur even though the sequences of the probe and target nucleic acid are not perfectly complementary, but instead include one or more mismatches. Conditions can be rendered less stringent by increasing the salt concentration and decreasing temperature. For example, a medium stringency condition includes about 0.1 to 0.25 M NaCl at temperatures of about 37° C. to about 55° C. Low stringency conditions include about 0.15M to about 0.9 M salt, at temperatures ranging from about 20° C. to about 55° C.

### VI. Exemplary Screening, Diagnostic and Classification Methods

### A. General Considerations

Certain methods that are provided involve determining the expression level of one or more of the differentially expressed genes in a test cell population with the expression level of the same genes in a control cell population, or comparing the expression profile for one sample with an expression profile determined for another sample. The level of expression of the differentially expressed nucleic acids can be determined at either the nucleic acid level or the protein level. Thus, the phrase “determining the expression level,” “preparing a gene expression profile,” and other like phrases when used in reference to the differentially expressed nucleic acids means that transcript levels and/or levels of protein encoded by the differentially encoded nucleic acids are detected. When determining the level of expression, the level can be determined qualitatively, but generally is determined quantitatively.

Based upon the sequence information that is disclosed herein, coupled with the nucleic acid and protein detection methods that are described herein and that are known in the art, expression levels of these genes can readily determined. If transcript levels are determined, they can be determined using routine methods. For instance, the sequence information provided herein (e.g., GenBank sequence entries) can be used to construct nucleic acid probes using conventional methods such as various hybridization detection methods (e.g., Northern blots). Alternatively, the provided sequence information can be used to generate primers that in turn are used to amplify and detect differentially expressed nucleic acids that are present in a sample (e.g., quantitative RT-PCR methods). If instead expression is detected at the protein level, encoded protein can be detected and optionally quantified using any of a number of established techniques. One common approach is to use antibodies that specifically bind to the protein product in immunoassay methods. Additional details regarding methods of conducting differential gene expression are provided infra.

Expression levels can be detected for one, some, or all of the differentially expressed nucleic acids that are listed in one or more of the tables. With some methods, the expression levels for only 1, 2, 3, 4 or 5 differentially expressed nucleic acids are determined. In other methods, expression levels for at least 6, 7, 8, 9 or 10 differentially expressed nucleic acids are determined. In still other methods, expression levels for at least 15, 20, 25, 30, 35, 40, 45, 50, 55, or 60 differentially expressed nucleic acids are determined. In yet other methods, all of the differentially expressed genes in one or more of the tables are determined.

Determination of expression levels is typically done with a test sample taken from a test cell population. As used herein, the term “population” when used in reference to a cell can mean a single cell, but typically refers to a plurality of cells (e.g., a tissue sample). Certain screening methods are performed with test cells that are “capable of expressing” one or more of the differentially expressed nucleic acids. As used in this context, the phrase “capable or expressing” means that the gene of interest is in intact form and can be expressed within the cell.

A number of the methods that are provided involve a comparison of expression levels for certain differentially expressed nucleic acids in a “test cell” with the expression levels for the same nucleic acids in a “control cell” (also sometimes referred to as a “control sample,” a “reference cell,” a “reference value,” or simply a “control”). Other methods involve a comparison between one expression profile and a baseline expression profile. In either case, the expression level for the control cell or baseline expression profile essentially establishes a baseline against which an experimental value is compared. The comparison of expression levels are meant to be interpreted broadly with respect to what is meant by: 1) the term “cell”, 2) the time at which the expression levels for test and control cells are determined, and 3) with respect to the measure of the expression levels.

So, for example, although the term “test cell” and “control cell” is used for convenience, the term “cell” is meant to be construed broadly. A cell, for instance, can also refer to a population of cells (e.g., a tissue sample), just as a population of cells can have a single member. The cell may in some instances be a sample that is derived from a cell (e.g., a cell lysate, a homogenate, or a cell fraction). In general samples can be obtained from various sources, particularly from lung cancers, or from body fluids of individuals suffering from lung cancer or at risk of lung cancer.

With respect to timing, comparison of expression levels can be done contemporaneously (e.g., a test and control cell are each contacted with a test agent in parallel reactions). The comparison alternatively can be conducted with expression levels that have been determined at temporally distinct times. As an example, expression levels for the control cell can be collected prior to the expression levels for the test cell and stored for future use (e.g., expression levels stored on a computer compatible storage medium).

The expression level for a control cell or baseline expression profile (e.g., baseline value) can be a value for a single cell or it can be an average, mean or other statistical value determined for a plurality of cells. As an example, the expression level for a control cell can be the average of the expression levels for a population of subjects. In other instances, the value for each expression level for the control cell is a range of values representative of the range observed for a particular population. Expression level values can also be either qualitative or quantitative. The values for expression levels can also optionally be normalized with respect to the expression level of a nucleic acid that is not one of the markers under analysis.

The comparative analysis required in some methods involves determining whether the expression level values are “comparable” (or similar”), or “differ” from one another. In some instances, the expression levels for a particular marker in test and control cells are considered similar if they differ from one another by no more than the level of experimental error. Often, however, expression levels are considered similar if the level in the test cell differs by less than 5%, 10%, 20%, 50%, 100%, 150%, or 200% with respect to the control cell. It thus follows that in some instances the expression level for a particular marker in the test cell is considered to differ from the expression level for the same marker in the control cell if the difference is greater than the level of experimental error, or if it is greater than 5%, 10%, 20%, 50%, 100%, 150% or 200%. In some methods, the comparison involves a determination of whether there is a “statistically significant difference” in the expression level for a marker in the test and control cells. A difference is generally considered to be “statistically significant” if the probability of the observed difference occurring by chance (the p-value) is less than some predetermined level. As used herein a “statistically significant difference” refers to a p-value that is <0.05, preferably <0.01 and most preferably <0.001. If gene expression is increased sufficiently such that it is different (as just defined) relative to the control cell or baseline, the expression of that gene is considered “up-regulated” or “increased.” If, instead, gene expression is decreased so it differs from the control cell or baseline value, the expression of that gene is “down-regulated” or “decreased.”

Comparison of the expression levels between test and control cells can involve comparing levels for a single marker or a plurality of markers (e.g., when expression profiles are compared). When the expression level for a single marker is determined, whether expression levels between the test and control cell are similar or different involves a comparison of the expression level of the single marker. When, however, expression levels for multiple markers are compared, the comparison analysis can involve two analyses: 1) a determination for each marker examined whether the expression level is similar between the test and control cells, and 2) a determination of how many markers from the group of markers examined show similar or different expression levels. The first determination is done as just described. The second determination typically involves determining whether at least 50% of the markers examined show similarity in expression levels. However, in methods where more stringent correlations are required, at least 60%, 70%, 80%, 90%, 95% or 100% of the markers must show similar expression levels for the expression levels of the group of markers examined considered to be similar between the test and control cells.

### B. Screening Methods

### 1. Exemplary Approaches

Monitoring changes in gene expression can provide certain advantages during drug screening and development. Often drugs are pre-screened for the ability to interact with a major target without regard to other effects the drugs have on cells. Often such other effects cause toxicity in the whole animal, which prevent the development and use of the potential drug. These global changes in gene expression provide useful markers for diagnostic, predictive and preventive uses as well as markers that can be used to monitor disease states, disease progression, and drug metabolism. Thus, these expression profiles of genes provide molecular tools for evaluating drug toxicity, drug efficacy, and disease monitoring.

Changes in the expression profile from a baseline profile (e.g., the data in Table 5) can be used as an indication of such effects. Those skilled in the art can use any of a variety of known techniques to evaluate the expression of one or more of the genes and/or gene fragments identified in the present application in order to observe changes in the expression profile in a cell or sample of interest. Comparison of the expression data, as well as available sequence or other information may be done by researcher or diagnostician or may be done with the aid of a computer and databases.

In some screening methods, compounds and molecules are screened to identify those that affect expression of a target gene or some other gene involved in regulating the expression of a target gene (e.g., by interacting with the regulatory region or transcription factors of a target gene). Compounds are also screened to identify those that affect the activity of such proteins (e.g., by inhibiting target gene activity) or the activity of a molecule involved in the regulation of a target gene.

So, for example, in some methods potential drug compounds are screened to determine if application of the compound alters the expression of one or more of the target genes identified herein. This may be useful, for example, in determining whether a particular compound is effective in treating asbestos-related lung cancer or other asbestos-mediated disease. In the case in which the expression of a gene in a cell suffered from asbestos-exposure is affected by the potential drug compound, the compound is indicated in the treatment of asbestos-related lung cancer or other asbestos-mediated disease. Similarly, a drug compound which causes expression of a gene which is normally down-regulated in a cell suffered from asbestos-exposure, may be indicated in the treatment of the same diseases.

According to the present invention, the target genes listed in Table 5 may also be used as markers to evaluate the effects of a candidate drug or agent on a cell suffered from asbestos-exposure. A candidate drug or agent can be screened for the ability to stimulate the transcription or expression of a given marker or markers (drug targets) or to down-regulate or inhibit the transcription or expression of a marker or markers. According to the present invention, one can also compare the specificity of a drug's effects by looking at the number of markers affected by the drug and comparing them to the number of markers affected by a different drug. A more specific drug will affect fewer transcriptional targets. Similar sets of markers identified for two drugs indicates a similarity in effect.

Some method are designed for identifying agents that modulate the levels, concentration or at least one activity of a protein(s) encoded by one or several genes in Table 5. Such methods or assays may utilize any means of monitoring or detecting the desired activity. Assays and screens can be used to identify compounds that are effective activators or inhibitors of target gene expression or activity. The assays and screens can be done by physical selection of molecules from libraries, and computer comparisons of digital models of compounds in molecular libraries and a digital model of the active site of the target gene product (i.e., protein).

The activators or inhibitors identified in the assays and screens may act by, but are not limited to, binding to a target gene product, binding to intracellular proteins that bind to a target gene product, compounds that interfere with the interaction between a target gene product and its substrates, compounds that modulate the activity of a target gene, or compounds that modulate the expression of a target gene or a target gene product.

Assays can also be used to identify molecules that bind to target gene regulatory sequences (e.g., promoter sequences), thus modulating gene expression. See, e.g., Platt (1994), J. Biol. Chem., 269:28558-28562.

### 2. Methods for Detecting Differential Gene Expression

Assays to monitor the expression of a marker or markers as defined in Table 5 may utilize any available means of monitoring for changes in the expression level of the target genes. As used herein, an agent is said to modulate the expression of a target gene if it is capable of up- or down-regulating expression of the target gene in a cell suffered from asbestos-exposure. The protein products encoded by the genes identified herein can also be assayed to determine the amount of expression. Any method for specifically and quantitatively measuring a specific protein or mRNA or DNA product can be used. However, methods and assays of the invention typically utilize PCR or array or chip hybridization-based methods when seeking to detect the expression of a large number of genes.

The genes identified as being differentially expressed in a cell suffered from asbestos-exposure may be used in a variety of nucleic acid detection assays to detect or quantify the expression level of a gene or multiple genes in a given sample. For example, traditional Northern blotting, dot blots, nuclease protection, RT-PCR, differential display methods, subtractive hybridization, and in situ hybridization may be used for detecting gene expression levels. Levels of mRNA expression may be monitored directly by hybridization of probes to the nucleic acids of the invention. If gene up- or down-regulation affects protein levels, proteins may be measured in all available methods, for example, Western blotting, ELISA, and immunohistochemistry. See, e.g., Sambrook et al, Molecular Cloning—A Laboratory Manual, Cold Spring Harbor Laboratory Press, Cold Spring Harbor, N.Y. (1989).

One of skill in the art will appreciate that an enormous number of array designs are suitable for the practice of this invention. The high density array will typically include a number of probes that specifically hybridize to the sequences of interest. See WO 99/32660 for methods of producing probes for a given gene or genes. In addition, in a preferred embodiment, the array will include one or more control probes.

### C. Diagnostic Methods

Methods for assessing whether a subject suffering from lung cancer has an asbestos-related lung cancer are also provided. These methods generally involve obtaining a sample from a subject having or suspected to have lung cancer and/or known or suspected to have been exposed to asbestos.

The diagnostic method of the present invention effectively identifies lung cancers associated with asbestos-exposure. The preferred method comprises steps of providing a sample of lung cancer cells taken from an individual suffering from lung cancer and detecting the type of allelic imbalance (AI) characteristic to asbestos-associated cancer in at least one of the following chromosomal regions of the lung cancer cells (see Table 3 and Table 6):
a) 19p13.3-p12;
b) 9q32-34.3;
c) 2p21-p16.3;
d) 16p13.3;
e) 22q12.3-q13.1; and
f) 5q35.3.

Asbestos-associated AI may extend beyond these regions. As shown in the experimental section, the presence of characteristic allelic imbalance (AI) in at least one of said regions indicates that the malignancy of the lung cancer cell is related to asbestos-exposure. The presence of AI in 2, 3, 4, or all of said regions confirms the significance of asbestos-mediated factors in development of the cancer.

Preferably, allelic imbalance is determined in the chromosomal region 19p13.3-p12, followed by the chromosomal regions 9q32-34.2 and 2p21-p16.3.

If AI occurs in all three above mentioned regions in a lung cancer case, there is 90% likelihood of this case being an asbestos-associated cancer. If AI occurs in none of these regions, the likelihood of the lung cancer case not being caused by asbestos is 98%.

As shown in Table 7, the presence of AI in the chromosomal region 19p13.3-p12 can be assessed by the use of the following microsatellite markers: 19S814, 19S883, 19S878, 19S424, 19S894, 19S216, 19S177, 19S1034, 19S873, 19S884, 19S916, 19S583, 19S535, 19S906, 19S221, 19S840, 19S917, 19S895, and 19S568, or by the use of any other polymorphic markers of this region. AI in 19p13.3-p12 can solely be used as a marker for asbestos-association of the lung cancer with 65% likelihood (Table 7.).

The allelic imbalance (AI) can be determined in multiple ways depending on the nature of the imbalance, i.e., loss or gain in asbestos-associated or non-asbestos-associated lung cancer. Preferable methods for the determination are, e.g., array technologies, loss of heterozygosity (LOH)-analyses, fluorescence in situ hybridization (FISH)-technology, and quantitative PCR, etc. Because AI may be, for example, a difference of only one copy of a certain chromosomal region between tumor and normal cells, detection of AI in cancer cells may require laser microdissection of cancer cells in order to avoid normal cell contamination in a sample. Laser microdissection is not needed, if AI, a deletion or amplification of chromosomal material is determined by FISH technology on tissue sections containing cancer cells. Specific arrays, e.g., oligo or SNP arrays, may be designed for the chromosomal regions that differentiate asbestos-associated lung cancers from those lung cancers without asbestos as a causal factor.

Moreover, expression level of individual or multiple genes as well as AI can be used to detect asbestos as a causal factor of a lung cancer case. The population of test cells is selected to include lung cancer cells from the subject. The expression level of the gene(s) is then preferably compared with the expression level of the same gene(s) in a control sample. The status of the control sample with respect to presence or absence of a lung cancer is preferably known (e.g., the control sample is from an individual not suffering from lung cancer but exposed to asbestos, or is preferably from an individual suffering from lung cancer but not exposed to asbestos). So, for example, if the control cell is representative of cells from an individual suffering from lung cancer but not exposed to asbestos, then similarity in expression level or expression profile between the test and control samples indicates that the subject does not have an asbestos-related disease. A difference in expression level or profile, in contrast, may indicate that the subject from whom the test sample was derived has an asbestos-related disease.

The detection of AI or gene expression characteristic to asbestos-associated lung cancer may also be used for early diagnosis, prediction, or prevention of lung cancer in asbestos-exposed individuals without the clinical condition of lung cancer but at risk to contract lung cancer. Tests for characteristic AI or gene expression at RNA or protein level may be applied to free nucleic acids or proteins deriving from abnormal cells in body fluids, e.g., sputum, bronchial washing, bronchoalveolar lavage, whole blood, plasma, or serum samples obtained from those individuals.

### VII. Devices for Detecting Differentially Expressed Nucleic Acids

### A. Customized Probe Arrays

### 1. Probes for Differentially Expressed Genes

The differentially expressed genes that are provided can be utilized to prepare custom probe arrays for use in screening and diagnostic applications. In general, such arrays include probes such as those described above in the section on differentially expressed nucleic acids, and thus include probes complementary to full-length differentially expressed nucleic acids (e.g., cDNA arrays) and shorter probes that are typically 10-30 nucleotides long (e.g., synthesized arrays). Typically, the arrays include probes capable of detecting a plurality of the differentially expressed genes of the invention. For example, such arrays generally include probes for detecting at least 2, 3, 4, 5, 6, 7, 8, 9 or 10 differentially expressed nucleic acids. For more complete analysis, the arrays can include probes for detecting at least 12, 14, 16, 18 or 20 differentially expressed nucleic acids. In still other instances, the arrays include probes for detecting at least 25, 30, 35, 40, 45 or all the differentially expressed nucleic acids that are identified herein.

### 2. Control Probes

### (a) Normalization Controls

Normalization control probes are typically perfectly complementary to one or more labeled reference polynucleotides that are added to the nucleic acid sample. The signals obtained from the normalization controls after hybridization provide a control for variations in hybridization conditions, label intensity, reading and analyzing efficiency and other factors that can cause the signal of a perfect hybridization to vary between arrays. Signals (e.g., fluorescence intensity) read from all other probes in the array can be divided by the signal (e.g., fluorescence intensity) from the control probes thereby normalizing the measurements.

Virtually any probe can serve as a normalization control. However, hybridization efficiency can vary with base composition and probe length. Normalization probes can be selected to reflect the average length of the other probes present in the array, however, they can also be selected to cover a range of lengths. The normalization control(s) can also be selected to reflect the (average) base composition of the other probes in the array. Normalization probes can be localized at any position in the array or at multiple positions throughout the array to control for spatial variation in hybridization efficiently.

### (b) Mismatch Controls

Mismatch control probes can also be provided; such probes function as expression level controls or for normalization controls. Mismatch control probes are typically employed in customized arrays containing probes matched to known mRNA species. For example, certain arrays contain a mismatch probe corresponding to each match probe. The mismatch probe is the same as its corresponding match probe except for at least one position of mismatch. A mismatched base is a base selected so that it is not complementary to the corresponding base in the target sequence to which the probe can otherwise specifically hybridize. One or more mismatches are selected such that under appropriate hybridization conditions (e.g. stringent conditions) the test or control probe can be expected to hybridize with its target sequence, but the mismatch probe cannot hybridize (or can hybridize to a significantly lesser extent). Mismatch probes can contain a central mismatch. Thus, for example, where a probe is a 20 mer, a corresponding mismatch probe can have the identical sequence except for a single base mismatch (e.g., substituting a G, a C or a T for an A) at any of positions 6 through 14 (the central mismatch).

### (c) Sample Preparation, Amplification, and Quantitation Controls

Arrays can also include sample preparation/amplification control probes. Such probes can be complementary to subsequences of control genes selected because they do not normally occur in the nucleic acids of the particular biological sample being assayed. Suitable sample preparation/amplification control probes can include, for example, probes to bacterial genes (e.g., Bio B) where the sample in question is a biological sample from a eukaryote.

The RNA sample can then be spiked with a known amount of the nucleic acid to which the sample preparation/amplification control probe is complementary before processing. Quantification of the hybridization of the sample preparation/amplification control probe provides a measure of alteration in the abundance of the nucleic acids caused by processing steps. Quantitation controls are similar. Typically, such controls involve combining a control nucleic acid with the sample nucleic acid(s) in a known amount prior to hybridization. They are useful to provide a quantitative reference and permit determination of a standard curve for quantifying hybridization amounts (concentrations).

### 3. Array Synthesis

Nucleic acid arrays for use in the present invention can be prepared in two general ways. One approach involves binding DNA from genomic or cDNA libraries to some type of solid support, such as glass for example. (See, e.g., Meier-Ewart, et al., Nature 361:375-376 (1993); Nguyen, C. et al., Genomics 29:207-216 (1995); Zhao, N. et al., Gene, 158:207-213 (1995); Takahashi, N., et al., Gene 164:219-227 (1995); Schena, et al., Science 270:467-470 (1995); Southern et al., Nature Genetics Supplement 21:5-9 (1999); and Cheung, et al., Nature Genetics Supplement 21:15-19 (1999), each of which is incorporated herein in its entirety for all purposes.)

The second general approach involves the synthesis of nucleic acid probes. One method involves synthesis of the probes according to standard automated techniques and then post-synthetic attachment of the probes to a support. See for example, Beaucage, Tetrahedron Lett., 22:1859-1862 (1981) and Needham-VanDevanter, at al., Nucleic Acids Res., 12:6159-6168 (1984), each of which is incorporated herein by reference in its entirety. A second broad category is the so-called “spatially directed” polynucleotide synthesis approach. Methods falling within this category further include, by way of illustration and not limitation, light-directed polynucleotide synthesis, microlithography, application by ink jet, microchannel deposition to specific locations and sequestration by physical barriers.

Light-directed combinatorial methods for preparing nucleic acid probes are described in U.S. Pat. Nos. 5,143,854 and 5,424,186 and 5,744,305; PCT patent publication Nos. WO 90/15070 and 92/10092; EP 476,014; Fodor et al., Science 251:767-777 (1991); Fodor, et al., Nature 364:555-556 (1993); and Lipshutz, at al., Nature Genetics Supplement 21:20-24 (1999), each of which is incorporated herein by reference in its entirety. These methods entail the use of light to direct the synthesis of polynucleotide probes in high-density, miniaturized arrays. Algorithms for the design of masks to reduce the number of synthesis cycles are described by Hubbel at al., U.S. Pat. No. 5,571,639 and U.S. Pat. No. 5,593,839, and by, Fodor et al., Science 251:767-777 (1991), each of which is incorporated herein by reference in its entirety.

Other combinatorial methods that can be used to prepare arrays for use in the current invention include spotting reagents on the support using ink jet printers. See Pease et al., EP 728, 520, and Blanchard, et al. Biosensors and Bioelectronics II: 687-690 (1996), which are incorporated herein by reference in their entirety. Arrays can also be synthesized utilizing combinatorial chemistry by utilizing mechanically constrained flowpaths or microchannels to deliver monomers to cells of a support. See Winkler et al., EP 624,059; WO 93/09668; and U.S. Pat. No. 5,885,837, each of which is incorporated herein by reference in its entirety.

### 4. Array Supports

Supports can be made of any of a number of materials that are capable of supporting a plurality of probes and compatible with the stringency wash solutions. Examples of suitable materials include, for example, glass, silica, plastic, nylon or nitrocellulose. Supports are generally are rigid and have a planar surface. Supports typically have from 1-10,000,000 discrete spatially addressable regions, or cells. Supports having 10-1,000,000 or 100-100,000 or 1000-100,000 regions are common. The density of cells is typically at least 1000, 10,000, 100,000 or 1,000,000 regions within a square centimeter. Each cell includes at least one probe; more frequently, the various cells include multiple probes. In general each cell contains a single type of probe, at least to the degree of purity obtainable by synthesis methods, although in other instances some or all of the cells include different types of probes. Further description of array design is set forth in WO 95/11995, EP 717,113 and WO 97/29212, which are incorporated by reference in their entirety.

### VIII. Kits

Kits containing components necessary to conduct the screening and diagnostic methods of the invention are also provided. Some kits typically include a plurality of probes that hybridize under stringent conditions to the different differentially expressed nucleic acids that are provided. Other kits include a plurality of different primer pairs, each pair selected to effectively prime the amplification of a different differentially expressed nucleic acid. In the case when the kit includes probes for use in quantitative RT-PCR, the probes can be labeled with the requisite donor and acceptor dyes, or these can be included in the kit as separate components for use in preparing labeled probes.

The kits can also include enzymes for conducting amplification reactions such as various polymerases (e.g., RT and Taq), as well as deoxynucleotides and buffers. Cells capable of expressing one or more of the differentially expressed nucleic acids of the invention can also be included in certain kits. Typically, the different components of the kit are stored in separate containers. Instructions for use of the components to conduct an analysis are also generally included.

The following examples are offered to illustrate certain aspects of the methods and devices that are provided; it should be understood that these examples are not to be construed to limit the claimed invention.

### EXPERIMENTAL SECTION

### Example 1

### Materials and Methods

Patients: We analyzed the copy number profiles of 14 malignant lung tumors from highly asbestos-exposed and 14 matched tumors from non-exposed individuals matched for age, gender, nationality and smoking history (Table 1). Asbestos exposure was estimated from work history obtained by personal interviews. In addition, the asbestos fiber count was measured by an electron microscopical analysis of lung tissue (Karjalainen 1993). The exposed group consisted of persons with a definite of probable exposure according to work history and the pulmonary asbestos fiber count higher than 5 million fibers/g dry weight. The asbestos fiber concentration of 2 to 5 million is thought roughly to represent a 2-fold increased risk of lung cancer due to asbestos-exposure (Karjalainen 1994, Consensus rep ort).

We analyzed 11 (5 exposed/6 non-exposed) adenocarcinomas (AC), 8 (4 exposed/4 non-exposed) squamous cell carcinomas (SCC), 5 (3 exposed/2 non-exposed) large cell lung carcinomas (LCLC) and 2 (1 exposed/1 non-exposed) each of adenosquamous carcinoma (AC/SCC) and small cell lung cancer (SCLC).

Tissue samples: Tissue samples were obtained during surgical operation for a tumorous lung lesion. The frozen tumor samples were cut to 4 μm sections for DNA isolation and for standard hematoxylin and eosin staining used to verify the tumor cell content (>50% requirement). DNA was isolated from tumor and reference (peripheral blood from 2 male donors) samples with QTAamp DNA Mini Kit (QIAGEN®, Valencia, Calif.).

Classical CGH: A classical CGH was performed on all 28 tumor samples according to Björkqvist et al. (1998). In brief, 1 μg of digested and labeled reference (TexasRed-5-dUTP and -dCTP) and tumor (FITC-5-dUTP and -dCTP) DNA was used for the hybridizations (NEN™ Life Science Products Inc., Boston, Mass.). The slides were hybridized over-night at 37° C. and washed according to standard protocols. The MetaSystems (MetaSystems GmbH, Altlussheim, Germany) CGH program, Isis (version 3) was used for analysis. Standard cut off thresholds at <0.85 for deletions, >1.17 for amplifications and >1.5 for high-level amplifications were used as described in Björkqvist et al (1998).

Array CGH: Array CGH analyses were conducted on 20 individual samples (11 exposed and 9 non-exposed, Table 1). Commercial cDNA microarrays (Human 1.0; Agilent Technologies, Palo Alto, Calif.) with 12 814 unique clones (97% map to named human genes) were used as described in Wikman et al. (2005). In brief, the hybridizations were performed with 5 μg of digested (25 U Alu1/25 U Rsa1) reference and tumor DNA, labeled (Cy3 dUTP-tumor, Cy5 dUTP-reference; Amersham Pharmacia Biotech, Piscataway, N.J., USA) with a random priming method (RadPrime DNA Labelling System, Gibco BRL, Gaithersburg, Md.). After hybridization at 65° C. overnight, the slides were washed, dried in a centrifuge and scanned with Agilent's DNA Microarray Scanner (G2565AA).

Data processing: The raw signal intensities were obtained from the arrays using the Feature Extraction software (Agilent Technologies). Measurements flagged as unreliable by the Feature Extraction software were removed from the subsequent analysis. Additionally, measurements defined as faulty by our own image analysis methods were removed. Our image analysis for detection of faulty measurement spots was performed as described previously (Ruosaari & Hollmén 2002) except that the spot foreground and background areas were obtained as a result of fitting two Gaussian distributions to each spot pixel neighborhood by using an expectation-maximization (EM)-algorithm. In this study, the quality assessment criteria for spots included in the subsequent analysis were as follows: 1) the size of the spot was larger than 15 pixels, 2) the intensity difference of the medians of the foreground and background pixels was at least 50 and 3) the median value of local background was less than 170. These quality assessment threshold values were obtained by first forming the respective distributions for good and faulty training spots labeled by an expert. The parameters were selected to minimize to probability of misclassification of the training spots (faulty spots being classified as faulty and faulty spots classified as good). After filtering, a proper signal with information of the gene locus could be obtained for 7730 to 9071 genes in the arrays. All arrays were normalized to have equal variance and mean Log 2 signal ratios.

Bioinformatics analysis: To identify exposure related aberrations, the array CGH data from individual patients were analyzed at group level by comparing gene copy numbers of the tumors of exposed and non-exposed patients. The identification of exposure-related areas was performed using 0.5-1 Mbp overlapping segments. First, the data were ordered according to the chromosomal location of the genes. Next, the genes within each segment were detected and the number of correctly classified asbestos-exposed and non-exposed patients was calculated.

The exposure-related aberrant regions were identified by means of hypothesis testing. In the two-tailed testing, the null hypothesis was set as “the segment's classification capability is not deviating” and the alternative hypothesis as “the segment's classification capability is deviating”. The number of correctly classified patients by the genes within each segment was used as a test statistic. The regions likely to be associated with exposure were found by the permutation test with 10 000 permutations using the empirical percentiles of 2.5 and 97.5 of the permutation distribution. Regions containing less than 5 genes were filtered away.

### Results and Discussion

Classical CGH: Typical patterns of aberrations were found with classical CGH for different histological types of lung cancer with SCLC having the most aberrations irrespective of exposure (Table 2) (online CGH database, available from: http://www.helsinki.fi/cmg/cgh_data.html). We detected in general more gains than losses with classical CGH, probably due to the fact that we did not use microdissected material. The most frequent changes in all patients were gains at 1q23-q24 (46%), 1q41 (64%), 2p23 (39%), 3q22-q23 (39%), 5p14-p15.1 (39%), 7p14 (25%), 8q24.1 (53%) and 20q13.1-q13.2 (68%), and losses at 9p23-p24 (14%) and 5 g (7%).

When comparing different histological types, all types except SCC showed slightly more aberrations in the exposed group (mean number of aberrations 6.7 and 3.2 in the exposed and non-exposed groups, respectively, in all histological types except SCC). SCC tumors had more aberrations in the non-exposed than in the exposed patients' samples due to two samples with 13 and 23 aberrations, respectively. The single amplification that seemed to differ significantly between the asbestos-exposed and the non-exposed groups in the classical CGH, was a minimal overlapping region in 2p23. This amplification was present in 57% ( 8/14cases) of the exposed and 14% ( 2/14cases) of the non-exposed patients' samples (p=0.025). In 7 out of 8 exposed cases the amplification included also 2p22 and in 4 cases 2p21.

Array CGH. As we did not find any clear changes, except the 2 p amplification, differing between the two groups with the classical CGH, we chose to analyze our array CGH results at the group level by comparing the signal log ratios in segments. This type of analysis does not require a priori knowledge of the type of aberrations in individual patients. Especially in this kind of comparative studies, where the aim is to detect changes associated with a certain factor, our choice of statistical method is beneficial due to synergetic reasons. The identification of aberrations from single array data separately is also possible, but small changes may not be detected due to the background noise on the arrays. In addition, when comparing several copy number data simultaneously, small changes common to a group of patients and significant low copy number changes may be detected.

Using this type of combined statistical analysis on the array CGH, we found 18 regions (1p36.12-p36.11, 1q21.2, 2p21-p16.3, 3p21.31, 4q31.21, 5q35.2-q35.3, 9q32, 9q33.3-34.11, 9q34.13-q34.3, 11p15.5, 11q12.3-q13.1, 11q13.2, 14q11.2, 16p13.3, 17p13.3-p13.1, 19p13.3-p13.11, 22q12.3-q13.1 and Xq28), which differed significantly in copy number between the two groups (Table 3). As expected from the classical CGH data, none of these regions harbored a high copy number change but either a low-level gain or a deletion. The choice of using combined analysis may not, however, fully compensate for the noise on the arrays caused by normal cell contamination. Therefore, there is a chance that, for instance, a gain in one group of patients is misinterpreted as loss in the other group. In addition, some of these loci seemed to be both amplified in one group and deleted in the other.

Most of the loci were very small (median size 1.76 Mbp), with the largest occurring on 19p13.3-p13.1 (18.53 Mbp). Two of the regions, 17p and 19p, were large enough (6.96 Mbp and 18.53 Mbp) to be detected with classical CGH, while the rest of the regions spanned 0.9-3.75 Mbp, which is usually too small to be detected by classical CGH (Forzan et al., 1997). With classical CGH, however, these two larger regions were not found. This method might have not detected these regions of loss because of normal cell contamination, for which our classical CGH results seemed to be more sensitive. Furthermore, these regions as well as the region 16p (3.14 Mbp) are so called problematic areas in classical CGH, which often give false positive or negative results due to hybridization artifacts (el-Rifai et al, 1997). Indeed, LOH analyses of both these regions have shown that lung tumors often harbor allelic imbalance at these loci (Girard et al, 2000). The region 9q34 (3.75 Mbp) has also been reported to be affected by LOH in lung cancer (Suzuki et al., 1998) and is also a problematic area in CGH (Larramendy et al., 1998).

Interestingly, the gain at 2p seemed to be specific for the exposed group based on both array and classical CGH results. A bit surprisingly, though, the minimal overlapping region in classical CGH was 2p23, whereas 2p21 was detected as altered in array CGH. However, with classical CGH in most cases, the 2p23 gain was larger and in 50% of the cases it contained 2p21. This quite large region could, thus, be target for further investigation, since a region homologous to the human 2p21-25 has previously been reported to be amplified in radon-induced rat lung tumors (Dano et al., 2000). Otherwise 2p amplifications have rarely been described in NSCLC. Similarly, the region 14q11.2 has never to our knowledge been reported to be altered in lung cancer, but it has been assumed to be involved in chromosomal aberrations (inversions and translocations) in the blood samples of a population exposed to prolonged low dose-rate 60Co gamma-irradiation (Hsieh et al., 2002). This could be interesting considering that radiation might cause similar aberrations to asbestos through the production of ROS (Leach et al., 2001).

Many of the significant regions found to separate the two groups have previously been implicated in lung carcinogenesis in general, including 1p36.1, 1q21.2, 3p21.31, 4q31.21, 5q35.2-q35.3, 9q34, 11p15.5, 17p13.3, 19p13 and 22q13 (http://www.helsinkili/cmg/cgh_data.html). However, a previous report has shown asbestos exposure to be significantly associated with 3p21 LOH (Marsit, 2004). Also, in vitro, asbestos fibers are mainly involved in causing breaks in chromosome 1 and 9 (Dopp & Schiffmann, 1998; Lohani et al., 2002).

The regions on the chromosomal arms 4q and 22q have been reported to be commonly lost also in mesothelioma (Björkqvist et al., 1998; De Rienzo, 2000), a cancer type very closely linked to asbestos exposure. Similarly, 11q13.1 contains the FOSLI (Fra-1) gene, which has been reported to be upregulated in transformed mesothelial cells after asbestos exposure (Shukla et al., 2004).

There are 125 listed fragile sites in the human genome and 11 of these coincide with the 18 potentially asbestos associated regions (p=0.08) in our results (Table 3). Fragile sites are predetermined chromosomal breakage regions, which experimentally can be demonstrated as site-specific gaps or breaks on metaphase chromosomes under conditions of replicative stress. They are known as a chromosomal expression of genetic instability and thus have been suggested to play a role in cancer. As an example, the FHIT gene at FRA3B (3p14.2) is often damaged in tumors and presumably acts as a tumor suppressor (Glover, 1998) as well as FRA16D (Finnis et al., 2005). Furthermore, in 11q13.2 a 700-kb deletion has recently been identified in cervical cancer, containing the fragile site FRA11A. This 700-kb region also lies almost completely within our region (Chr 11:65,886,588-67,191,050 bp) (Zainabadi et al., 2005). The fragile sites are, however, mostly mapped according to G-banding methods and we cannot, at a higher resolution, conclude whether our regions are exactly the same as the fragile site regions, except for 11q13.2.

In conclusion, to reveal the possible aberrations related to asbestos exposure in the array data, we chose to carry out the data-analysis using the combined array dataset. By using this method we could detect for the first time several, mostly small chromosomal regions that differed in DNA copy number between these two groups of patients' lung tumors. The aberrations were either low copy number gains or losses with no high copy number amplifications. Previous studies have implied that smoking makes the genetic system of the cells more vulnerable to the deleterious effects of asbestos (26, 27). This evidence is in agreement with our classical CGH results, in which the same complex patterns of aberrations were generally found in both groups with just slightly more aberrations in the exposed group. Furthermore, our array CGH results showed that many of these sites coincided with fragile sites implying that smoking and asbestos fibers may preferentially cause aberrations at fragile sites. To conclude, we report for the first time gene copy number aberrations related to asbestos-exposure. Further verifying analysis, using for example expression data, is needed to show whether these regions are specific and harbor putative target genes.

### Example 2

### Materials and Methods

### Patient Material

All patients were males of Finnish Caucasian origin with histologically confirmed primary lung cancer and no previous malignancies. The samples for gene expression analysis consisted of lung tumor and corresponding normal lung samples from 14 highly asbestos-exposed and from 14 non-exposed patients (Table 4). In subsequent fragment analyses for allelic imbalance 15 additional tumors from non-exposed patients and 8 tumors from patients with occupational exposure to asbestos (intermediate exposure group) were analyzed (Table 4). All the tumors were classified according to the latest WHO classification.

Detailed information of the patients' work history as well as of their smoking habits and survival data were recorded. The level of asbestos exposure was estimated both by work history and by measurement of the pulmonary asbestos fiber concentration by scanning electron microscopy with energy dispersive spectrometry (Karjalainen et al., 1993). Only patients with a definite or probable occupational exposure to asbestos (Karjalainen et al., 1993), and more than 5 million fibers per gram of dry lung tissue were included in the heavy exposure group. Patients with a concentration between 1 and 5 million fibers per gram were classified as intermediately exposed. A minimum of 1 million fibers per gram of dry lung tissue is usually considered as a sign of occupational exposure to asbestos (Karjalainen et al., 1993). A 2-fold risk of lung cancer is related to fiber levels of 2-5 million per gram of dry lung (Karjalainen et al., 1994; Consensus report, 1997).

All patients were personally interviewed and their consent to take part in the study and to use their tissue was obtained. The Ethical Review Board for Research in Occupational Health and Safety, Helsinki and Uusimaa Health Care District, has approved the study protocol (75/E2/2001).

cDNA Microarrays

RNA was isolated with Ultraspec™ RNA isolation system from tumor and adjacent normal peripheral lung tissue for each patient as described in (Wikman, 2002). Each tumor sample was cut in a cryotome and the tumor content of each sample was verified by HE staining. Only samples with more than 50% tumor cells were chosen for the analysis. After initial isolation RNA was purified further with Qiagen RNase Minikit column purification. The quality of RNA was assessed with 2100 Bioanalyzer (RNA Nano Labchip, Agilent Technologies, Palo Alto, Calif.) and quantified by spectrophotometer.

Gene expression profiling was conducted using Affymetrix HU133A GeneChips (Affymetrix, Santa Clara, Calif.) with 6 μg of total RNA. The RNA was converted to cDNA by one-cycle cDNA Synthesis Kit (Invitrogene, Carlsbad, Calif.), purified, and converted to labeled cRNA (Enzo, Farmingdale, N.Y.) according to Affymetrix recommendations. The fragmented cRNA was hybridized for 16 hours. Washing, staining, and scanning of the slides were performed according to the standard Affymetrix protocols. Hybridizations on Affymetrix chips were carried out with tumor and normal lung RNA samples from each of the 28 patients.

### Data Analysis of the Gene Expression Data

All slides were scaled for the value 100. The tumor chips were scaled with respect to their matched normal lung chips. For the three cases with a missing normal lung result, the mean signal of the samples from the same exposure-group was used instead as a reference. Genes that were present (Affymetrix p-value <0.04) in at least one third of the exposed or non-exposed samples were included in the analyses. Next, the data were log 2 transformed and Lowess normalized.

A two-step analysis model was used to detect differentially expressed genes and to identify the smallest set of genes that could distinguish the exposed group from the non-exposed group. We used a supervised classification method similar to that described by van't Veer et al. (van't Veer, 2002). As the first step, AUROC (ROC) analysis model (Kettunen, 2004) was chosen due to similar size of the two exposure groups. Genes with ROC values larger than 0.4, or smaller than 0.6, and with p-value smaller than 0.4 were included in the subsequent analyses.

In the second step, a correlation coefficient for the gene expression and exposure status (asbestos-exposed versus non-exposed) was calculated for each gene. As we were primarily interested in the differences between the tumors of asbestos-exposed and non-exposed patients and, in order to minimize the effect of variation in gene expression between individual normal lung tissue samples, the data were resealed before conducting the correlation analysis. To emphasize the differences between the asbestos-associated and non-associated tumors, the signals of the asbestos-associated tumors were scaled by the median signal of the non-associated tumors and the signals of the non-associated tumors by the median signal of the asbestos-associated tumors.

The genes were rank-ordered according to the absolute value of the correlation coefficient. To optimize the number of genes needed for the correct classification of tumors, the genes were added sequentially according to their rank-order, and the number of correctly classified patients was determined. A “leave-one-out” method was used for cross-validation.

### Analysis of Combined Gene Expression and DNA Copy Number Data

We have also described the aberration (amplifications and deletions) profiles of lung tumors of asbestos-exposed versus non-exposed patients with array CGH analysis (see Example 1). To further define the exposure-related areas, we in the current study combined the gene expression and copy number profiles of the tumors from the same patients.

Identification of the chromosomal areas with exposure-associated changes was performed by comparing the gene expression ratios of the exposed to the non-exposed in overlapping segments of 0.5-1 Mbp. The differential regions were identified by means of hypothesis testing. The number of patients correctly classified by the gene expression ratios of each gene was calculated and, as a test statistic, an average classification capability of the segment was used. The regions found in this analysis were compared to the regions found to have exposure-associated copy number changes. The regions that were detected both in the expression and copy number data sets were considered prominently interesting.

### Fragment Analysis for Detection of Allelic Imbalance

The samples used in fragment analyses included both microdissected and not microdissected DNA specimens. The original 28 tumor samples from highly asbestos-exposed and non-exposed patients were macrodissected, whereas microdissection was used to obtain DNA from the additional 23 patient samples. However, because ambiguous results were obtained from the 28 patient samples that were not microdissected (with several markers the ratio of the peak heights in tumor and normal alleles was close to 1.5), the experiments were repeated with the corresponding microdissected material.

Microdissection was performed using an Arcturus Veritas instrument on 9 μm tissue sections stained with 1% toluidine blue-0.2% methylene blue solution. Laser capture microdissection (LCM) technology was utilized to harvest cancer cells from heterogeneous tumor tissues. DNA was isolated using a PicoPure™ DNA Extraction Kit (Arcturus) according to the manufacturer's instructions.

Allelic balance of the chromosomal region 19p13.3-p12 (chr19:550811-22287245 bp; 22.29 Mbp) was assessed using 19 microsatellite markers with approximate coverage of 22 Mbp. FAM or HEX end-labeled primer pairs were used to amplify the di- or trinucleotide-repeat fragments of 80-300 by in length. The primer sequences for the markers were obtained from the data bases of the National Center for Biotechnology Information and synthesized at TIB MOLBIOL Syntheselabor GmbH. The target sequences were amplified by PCR in a volume of 5 μl or 10 μl containing 200 μM dNTPs, 700 nM of each primer, 1×PCR Buffer containing 15 mM MgCl₂, 0.13 or 0.25 units of HotStarTaq DNA Polymerase (Qiagen), respectively, and 2.5-25 ng of genomic DNA. An initial 10 min 95° C. denaturation step was followed by 35 cycles of 95° C. for 40 s, 40 s at the optimized annealing temperature, and 72° C. for 1 min. The PCR products were then analyzed with a 3100-Avant Genetic Analyzer (Applied Biosystems).

The determination of allelic imbalance (AI) was performed for heterozygous markers by calculating the ratio of the peak heights of the tumor and normal alleles. Alleles were defined as the two highest peaks within the expected size range. Ratios of 1.5 or higher were scored as AI. Microsatellite instability (MSI) was defined by the presence in the tumor DNA of novel peaks with the size that differed from normal DNA by an integer number of repeat units. Additionally, the mononucleotide repeat BAT-26 was used to test its correlation with the MSI phenotypes in lung cancer. This marker has previously been used to reveal a high-frequency MSI phenotype of sporadic colorectal and gastric cancers with 99.4-100% accuracy (Hoang, 1997).

### Results

### Gene Expression Profiles

ROC analysis was carried out using the gene expression data to detect genes that best separated the 14 highly asbestos-exposed from the non-exposed patients. 12 865 genes were included in the first ROC analysis (inclusion criterion was the presence of a signal in at least ⅓ of the patients from either exposure group). The genes were ordered according to their ROC and p-values.

A crude unsupervised, hierarchical clustering algorithm based on genes with the highest ROC values (<0.4 or >0.6 and with p-value smaller than 0.4) allowed us to cluster the 28 tumours into two groups on the basis of their exposure (data not shown). The clear division of tumours in exposed and non-exposed tumours suggested that the tumours can be divided into these two types on the basis of about 6000 gene transcripts.

Next, the correlation coefficient of gene expression with exposure status (asbestos exposed versus non-exposed) was calculated for each gene. To identify the smallest set of genes that could distinguish the two tumour groups, the genes were rank-ordered according to the absolute value of the correlation coefficient. The identification of exposure associated genes revealed 47 genes with Pearson's correlation coefficient larger than 0.79 or smaller than −0.79. We note that our choice of reference (median signals of asbestos-associated tumors were scaled by the median signals of non-associated tumors and vice versa) gives rise to the relatively high correlation coefficient, but similar results are obtained when median signals of normal tissue of each group was used as a reference. 38 of the 47 top genes are identical using both references. Only single genes with such magnitude of correlation coefficient could be detected after random permutation of the data. The functional annotation for this small set of top genes (47) did not give clear overrepresentation of a specific function or chromosomal localization. Hierarchical clustering results obtained for these 47 genes are shown in Table 5.

Combination of Gene Expression Profiles with DNA Aberration Profiles

The identification of exposure related areas with expressional changes revealed 34 areas (areas within 5 Mbp were combined) on which the asbestos exposed patients differed from the non-exposed ones (data not shown). The detection of the areas was performed by comparing the gene expression data of the two patient groups to each others in 0.5-1 Mbp segments similarly as was done for the CGH array (see Example 1). Areas with exposure related changes were identified by means of permutation testing with 5% confidence intervals. To identify loci both with exposure associated mRNA (Affymetrix) and DNA level (CGH array) changes, results from these two data-analyses were combined. Six areas were common in the two analyses, namely 2p21-p16.3, 3p21.31, 5q35.2-q35.3, 16p13.3, 19p13.3.-13.11, and 22q12.3-q13.1 (Table 6). The data suggests that 2p21 could be simultaneously amplified in the exposed and deleted in the non-exposed patient samples whereas 3p21.3-p21.1, 5q35.3, and 22q13.1 seem to be deleted among the exposed group of patients. The largest significant region was detected on chromosome 19p13.3-19p13.1. Most exposed patients showed a deletion and down-regulation of genes in this region whereas some of the non-exposed patients showed a possible gain.

### Fragment Analysis on 19p13.3-12

Fragment (LOH) analysis was carried out to verify the existence of exposure associated changes on p-arm of the chromosome 19 and to reveal the extent of the aberration. 19 microsatellite markers spanning 22.3 Mbp region on 19p13.3-p12 were used (Table 7). 79% ( 11/14) of the exposed and 45% ( 13/29) of the non-exposed patients (p=0.02) were found to be carriers of allelic imbalance (AI) on the 19p region. Additionally AI was detected in 75% ( 6/8) of the moderately exposed patients (Table 7). The patients in whom AI was detected were in good accordance with the results indicated by the CGH array. The AI degree for individual markers ranged between 50-90% in exposed, 40-100% in intermediately exposed, and 20-50% in non-exposed patients (only informative markers taken into account). When focusing into the differences in the frequency of AI in individual markers, differential separation was observed between the 19 markers studied. The frequency of chromosomal alterations was significantly higher in 10/19 of the markers in the tumour samples from the asbestos exposed patients compared with the non-exposed patients.

Additionally, 10% ( 3/29) of the non-exposed patients were found to have microsatellite instability (MSI) ranging throughout the region studied. When further assessing MSI with the colon MSI marker BAT-26, two of the three cases (149 and 62) showing high instability in the individual markers also showed instability in this marker. Additionally, patients 11 and 143 that both had a single marker showing MSI on the 19p region also had instability in the marker BAT-26.

### Discussion

To bring insight to the deregulated genes associated with asbestos related lung cancer, we performed a combined cDNA and CGH microarray screening analysis on 28 primary lung tumours. Highly asbestos exposed patients were compared with non-exposed lung tumours and differences in both the gene expression level and copy number changes between the two groups were described. One of the most interesting regions, 19p, was further verified with enlarged number of patients.

We used a two step data analysis procedure for the gene expression results and found a set of 47 genes that correctly classified patients into asbestos exposed and non-exposed groups. Hierarchical clustering analyses on these 47 genes show a clear division of the two exposure groups. The separation is independent of histological lung cancer type. This 47 marker gene set included genes representing a wide variety of functions with no single pathway over-represented. Even though several of these genes are currently fairly unknown, quite a few of them have been found altered in various different tumour types. These include the genes detected to be upregulated WFDC2, TDE1, and SLC6A15 and downregulated RUNX1, ATM, and UVRAG in the exposed patients. The WFD2 (HE4) has been shown to be a biomarker for ovarian carcinoma (Hellstrom, 2003) and SLC6A15 has been shown to be upregulated in colorectal cancer (Gupta, 2005). The TDE1 gene has been shown to be upregulated in lung cancer cell lines (Bossolasco, 1999). The UVRAG gene, which was downregulated among the exposed was recently shown to be mutated in colon cancer (Ionov, 2004), while ATM is known to be silenced in lung cancer by promoter hypermethylation (Safar, 2005). Whereas the homologous RUNX3 gene has been shown to be downregulated by methylation in lung tumours (Li, 2004), RUNXI translocations, mutations and methylation has been described in mainly various leukaemia, but also lately in gastric cancer (Sakakura, 2005); Blyth, 2005).

Adducin—a substrate of proteinkinase C (PKC)— has been associated with asbestos exposure. The PKC signal transduction pathway is suggested to be one of the main signalling pathways to be activated after asbestos exposure (Shukla, 2003). Indeed, mice that have inhaled asbestos show an increased expression of adducin in the alveolar type II lung epithelial cells (Lounsbury, 2002). Similar to these findings adducin was found to be upregulated among the exposed patients in this study.

A recent study showed, it is extremely difficult to find stable and reliable molecular signatures from microarray data, even when the data sets are large (Michiels, 2005). We are, therefore, aware that by doing this type of analysis with thousands of genes but few patients, one has a big chance of finding false positive results. As a result, as we had seen in our previous study with classical and CGH array data that lung cancer can be separated according to their DNA copy number profiles (see Example 1), we decided to investigate whether these specific chromosomal regions could be correlated with gene expression changes. Potential good markers with biological relevance are those aberrations that have an influence on the gene expression. Indeed with this method we could find six chromosomal regions that were simultaneously changed at both DNA and RNA level and seemed to be specific for one group of tumors. Interestingly, four of the regions seemed to be deletions in the exposed group.

Chromosome 3p, 5q, 19p and 22q aberrations which we found significantly associated with asbestos exposure have all been previously detected in lung carcinogenesis in general. However, recently an association of loss of 3p and asbestos exposure was described, showing that even though both groups do show the aberrations the frequency of 3p is significantly higher in the exposed group (Marsit, 2004). Furthermore, the region on 22q has been reported to be commonly lost in mesothelioma, a cancer type very closely linked to asbestos exposure (De Rienzo, 2000). Whereas 2p amplifications have rarely been described in lung tumors, it has been shown since a region homologous to the human 2p21-25 has previously been reported to be amplified in radon-induced rat lung tumors (Dano, 2000). 16p13.3 contains the gene TSC2 which has been described to be affected by LOH in 29% of lung adenocarcinomas (Takamochi, 2004) and the gene NTHL1, involved in 8oxoG repair, which has been shown to have lower expression in lung cancer compared to normal lung tissue (Radak, 2005)

In this study, the association of one of the possibly asbestos related chromosomal regions—19p13—was further verified. LOH of 19p is common in lung cancers (Sanchez-Cespedes 2001), but its relation with asbestos exposure has not been previously studied. Here fragment analysis was carried out to reveal AI. As expected, the chromosomal changes were not only limited to the exposed patients but they were significantly more common among the exposed than non-exposed (p=0.02). AT in the 19p13 region was detected in 79% exposed, 75% intermediate exposed and 45% non-exposed patients indicating that exposure seems to work in favour of the aberration of this area. The markers with best separation are spread out through the 19p region indicating that there may not specific asbestos exposure related hotspots within the area but rather an association with asbestos and imbalance of the whole chromosomal arm.

Noteworthy is that two genes previously reported to be inactivated in lung cancer reside proximal to some of the most significantly distinguishing markers. The inactivation through mutations and LOH of the tumour suppressor gene STK11/LKB1 located next to the marker D19S883 has been found to occur in 30% of sporadic lung adenocarcinoma (Sanchez-Cespedes, 2002). Additionally, the BRG1/SMARCA4 gene located close to the marker D19S906 has been implied to have a role in lung tumorigenesis (Medina, 2005). The SMARCA4 protein has been reported to be lost in about 10% of the lung primary tumours (Reisman, 2003).

Fragment analysis does not, however, differentiate between allelic gain and loss and thus the changes in markers may only be reported as AI. Our array CGH results do, however, suggest that there are both losses and gains on the 19p13 region in the tumour samples, gains especially among the non-exposed patient samples. Therefore the association of this region with exposure may be underestimated by our current results Additional studies should thus be carried out by means of e.g. quantitative PCR to gain better insight into the nature of changes occurring in this region. Such studies are expected to strengthen the relatedness of 19p aberration, especially loss, to asbestos exposure.

In conclusion, by combining different high trough-put methods we show for the first time that asbestos exposed lung cancer patients have a distinct gene expression profile with certain chromosomal regions such as 19p significantly associated with the exposure.

### Example 3

### Methods for Detection of the Aberration Profile of Asbestos-Related Lung Cancer

The present aberrations can be detected with following methods for example: array CGH based on oligo or BAC clone chips; SNP arrays; in situ hybridization (FISH, CISH) probe sets; fragment analysis for allelic imbalance; quantitative gene-dose PCR.

9q32-q34

Table 8 shows the fragment analysis results on allelic imbalance on 9q31.3-q34.3 in adenocarcinomas and other histological lung tumor types of asbestos-exposed and non-exposed patients. In general, more allelic imbalance was found in asbestos-exposed than in non-exposed patients' tumors. Tests for allelic imbalance have been carried out with microdissected tumor tissue.

Three FISH probes have been tested on lung tumor sections: BAC probes RP11-10i9, RP11-375D21, and RP11-100C15. The results obtained with these three probes are shown in Table 9. More deletions and gains were detected in asbestos-exposed than in non-exposed patients' tumors in all histological types with the BAC probe RP11-375D21, whereas with two other probes all other histological types except adenocarcinomas of asbestos-exposed had more aberrations.

Table 10 shows the combination of allelic imbalance in 19p and 9q with BAC probe RP11-375D21. Combination improves the specificity for identification of asbestos-related and non-related lung tumors.

2p16-p21

Table 11 shows the allelic imbalance on 2p16-p21. Fourteen asbestos-exposed and 14 non-exposed patients' tumors were studied by fragment analysis with microsatellite markers. Results are given for markers with minimum 6 informative cases in each group. Tests for allelic imbalance have been carried out with microdissected tumor tissue. More allelic imbalance was detected in asbestos-exposed than in non-exposed patients' tumors.

16p13.3

Table 12 shows allelic imbalance on 16p13.3 detected by fragment analysis with microsatellite markers. More allelic imbalance was detected in asbestos-exposed than in non-exposed patients' tumors. Tests for allelic imbalance have been carried out with microdissected tumor tissue.

Similarly with 9q results, adenocarcinomas of the exposed patients differed from other histological tumor types.

5q35.3

Table 13 shows allelic imbalance in 5q35.3 in lung tumors of asbestos-exposed and non-exposed patients. Tests for allelic imbalance have been carried out with microdissected tumor tissue. Fragment analysis for allelic imbalance did not show clear differences between exposed and non-exposed patients' tumors. However, array CGH results given on Table 14 warrant further investigations of this region.

### Example 4

The aim of this example was to investigate whether asbestos-exposure causes a specific gene expression profile that correlates with the previously detected asbestos-associated genomic aberration profile. By combining the gene expression data with the comparative genomic hybridization (CGH) array data, we were able to detect six distinct chromosomal regions that harbor both gene expression and DNA level changes. One of these, 19p13.3-19p13.1 was further characterized for allelic imbalance by using 19 microsatellite markers on lung carcinomas from 62 male patients chosen on the basis of their present or absent asbestos-exposure determined by the work histories and pulmonary asbestos fiber counts.

### Materials and Methods

### Patient Material

All patients were of Finnish Caucasian origin with histologically confirmed primary lung cancer and no previous malignancies. The samples for gene expression analysis consisted of lung tumor and corresponding normal lung samples from 14 heavily asbestos-exposed and from 14 non-exposed patients (Table 17). The subsequent microsatellite analyses for allelic imbalance in 19p were done on the original set of 28 cases and on 34 additional lung cancer cases chosen on the basis of the level of asbestos-exposure: 11 heavily asbestos-exposed, 8 moderately occupationally asbestos-exposed, and 15 non-exposed lung cancer cases (Table 18). The Ethical Review Boards for Research in Occupational Health and Safety and the Coordinating Ethical Review Board, Helsinki and Uusimaa Hospital District, have approved the study protocols (223/E0/2005 and 75/E2/2001). The National Agency for Medicolegal Affairs has given the permission to use diagnostic samples for the research purpose (4476/33/300/05), and the Ministry for Social Affairs and Health has permitted the collection of patient information for research (STM/2474/2005).

In all cases the level of asbestos exposure was estimated both by work history and by measurement of the pulmonary asbestos fiber concentration (Karjalainen et al., 1993). Only patients, who had both a definite or probable occupational exposure history to asbestos according to an interview, and more than 5 million fibers per gram of dry lung tissue were included in the heavy exposure group. Patients with a concentration between 1 and 5 million fibers per gram were classified as moderately exposed. A minimum of 1 million fibers per gram of dry lung tissue is usually considered as a sign of occupational exposure to asbestos (Karjalainen et al., 1993). In the non-exposed group were included only patients in whom neither the exposure history nor the pulmonary fiber count indicated an exposure to asbestos.

### Expression Microarrays

RNA was isolated with Ultraspec™ RNA isolation system from tumor and adjacent normal peripheral lung tissue for each patient as described in (Wikman et al., 2002). The quality of RNA was assessed with 2100 Bioanalyzer (RNA Nano Labchip, Agilent Technologies, Palo Alto, Calif.) and quantified by spectrophotometer.

Gene expression profiling was conducted using Affymetrix HU133A GeneChips (Affymetrix, Santa Clara, Calif.) with 6 μg of total RNA. The RNA was converted to cDNA by one-cycle cDNA Synthesis Kit (Invitrogene, Carlsbad, Calif.), purified, and converted to labeled cRNA (Enzo, Farmingdale, N.Y.) according to Affymetrix recommendations. The fragmented eRNA was hybridized for 16 hours. Washing, staining, and scanning of the slides were performed according to the standard Affymetrix protocols. Hybridizations on Affymetrix chips (HU133A) were carried out with tumor and normal lung RNA samples from each of the 28 patients.

### Data Analysis of the Gene Expression Data

Affymetrix Analysis Suite version 5 (MASS) was used to scale the arrays for the target value of 100 and to define the absent/present calls. Only samples with a background of 40-70 and house keeping control signal ratios (5′ to 3′ prime end transcript ratio) close to one were included in data analysis. As a result of these criteria, 3 normal lung samples were excluded from the study.

Chips of matched normal lung samples were used as a reference for the tumor chips. For the three cases with a missing normal lung result, the mean signal of the samples from the same exposure-group was used instead as a reference. Genes that were present (Affymetrix p-value <0.04) in at least one third of the exposed or non-exposed samples were included in the analyses. Next, the data were log 2 transformed and Lowess normalized. A two-step analysis model was used to detect differentially expressed genes and to identify the smallest set of genes that could distinguish the exposed group from the non-exposed group. As the first step, AUROC(ROC) analysis model (Kettunen et al., 2004) was chosen due to similar size of the two exposure groups. Genes with ROC values smaller than 0.4, or larger than 0.6, and with p-value smaller than 0.4 were included in the subsequent analyses.

In the second step, a correlation coefficient for the gene expression and exposure status (asbestos-exposed versus non-exposed) was calculated for each gene. As we were primarily interested in the differences between the tumors of asbestos-exposed and non-exposed patients and, in order to minimize the effect of variation in gene expression between individual normal lung tissue samples, the data were resealed before conducting the correlation analysis. To emphasize the differences between the asbestos-associated and non-associated tumors, the signals of the asbestos-associated tumors were scaled by the median signal of the non-associated tumors and the signals of the non-associated tumors by the median signal of the asbestos-associated tumors.

The genes were rank-ordered according to the absolute value of the correlation coefficient. To optimize the number of genes needed for the correct classification of tumors, the genes were added sequentially according to their rank-order, and the number of correctly classified patients was determined. A “leave-one-out” cross-validation method was used to assess the reliability of the classification.

### Analysis of Combined Gene Expression and DNA Copy Number Data

Identification of the chromosomal areas with exposure-associated gene expression changes was performed by comparing the gene expression ratios of the exposed to the non-exposed locally. The chromosomes were divided in overlapping segments of 0.5-1 Mbp and each segment was tested for differential expression. The differentially expressed regions were identified by means of hypothesis testing. The number of patients correctly classified by the gene expression ratios of each gene was calculated and, as a test statistic, an average classification capability of the segment was used. The regions found in this analysis were compared to the regions found to have exposure-associated copy number changes. The regions that were detected both in the expression and copy number data sets were considered prominently interesting.

### Microsatellite Analysis for Detection of Allelic Imbalance

Microsatellite analysis was used as a validation method for confirming the presence of allelic imbalance. The samples used in microsatellite analyses included both microdissected and not microdissected DNA specimens. The original 28 tumor samples from heavily asbestos-exposed and non-exposed patients were macrodissected, whereas microdissection was used to obtain DNA from the additional 34 patient samples. Samples for microsatellite analysis were from freshly frozen tissue in 52 cases and from paraffin-embedded tissue in 10 cases.

Microdissection was performed using an Arcturus Veritas instrument on 9 μm tissue sections stained with 1% toluidine blue-0.2% methylene blue solution. Laser capture microdissection (LCM) technology was utilized to harvest cancer cells from heterogeneous tumor tissues. DNA was isolated using a PicoPure™ DNA Extraction Kit (Arcturus) according to the manufacturer's instructions.

Allelic balance of the chromosomal region 19p13.3-13.1 (chr19:550811-22287245 bp; 22.29 Mbp) was assessed using 5-19 microsatellite markers with approximate coverage of 22 Mbp. FAM or HEX end-labeled primer pairs were used to amplify the di- or trinucleotide-repeat fragments of 80-300 by in length. The primer sequences for the markers were obtained from the data bases of the National Center for Biotechnology. The target sequences were amplified by PCR and the PCR products were then electrophorized with a 310 or 3100-Avant Genetic Analyzer (Applied Biosystems).

GeneMapper Analysis Software version 3.5 (Applied Biosystems) was used to study the lengths of the allele fragments. The alleles were defined as the two highest peaks within the expected size range. The determination of allelic imbalance (AI) was performed for heterozygous markers by calculating the ratio of the peak heights of the tumor and normal alleles. Ratios of 1.5 or higher were scored as AI. The criterion based on which AI-carriers were determined was that at least 25% of the informative microsatellite markers had to be AI-positive. The mononucleotide repeat BAT-26 was used to test its correlation with the MSI phenotypes in lung cancer. This marker has previously been used to reveal a high-frequency MSI phenotype of sporadic colorectal and gastric cancers with 99.4-100% accuracy (Hoang et al., 1997).

### Results

### Gene Expression Profiles

ROC analysis was carried out using the gene expression data to detect genes that best separated the lung tumors of 14 heavily asbestos-exposed patients from the tumors of 14 non-exposed patients. 12 865 genes were included in the first ROC analysis (inclusion criterion was the presence of a signal in at least ⅓ of the patients in either exposure group).

A crude supervised algorithm based on genes with the highest ROC values (<0.4 or >0.6, and with p-value smaller than 0.4) allowed us to cluster the 28 tumors into two groups on the basis of the exposure of the patients (data not shown). The clear division of the tumors according to the exposure category of the patients suggested that the tumors can be divided into these two types on the basis of about 6000 gene transcripts.

Next, the correlation coefficient of gene expression with the exposure status of the patient (asbestos-exposed versus non-exposed) was calculated for each gene. To identify the smallest set of genes that could distinguish the two tumor groups, the genes were rank-ordered according to the absolute value of the correlation coefficient. The identification of exposure-associated genes revealed 47 genes with Pearson's correlation coefficient larger than 0.8 or smaller than −0.8. We note that our choice of reference (the median signal of the non-associated tumors for the asbestos-associated tumors and the median signal of the asbestos-associated tumors for the non-associated tumors) gives rise to the relatively high correlation coefficient, but similar results are obtained when median signals of normal tissue of each group was used as a reference. 38 of the 47 top genes are identical with both references. Only single genes with similar magnitude of correlation coefficient could be detected after random permutation of the data. The functional annotation for this small set of top genes (47) did not show clear overrepresentation of a specific function or chromosomal localization.

Combination of Gene Expression Profiles with DNA Aberration Profiles

The identification of exposure-related areas with expressional changes revealed 34 areas (areas within 5 Mbp were combined) on which the tumors of asbestos-exposed patients differed from the tumors of non-exposed patients (data not shown). The detection of the areas was performed by comparing the gene expression data of the two tumor groups to each other in 0.5-1 Mbp segments similarly as was done for the CGH array (Nymark et al., 2006). Areas with exposure-related changes were identified by means of permutation testing. The regions were declared significant if the observed expressional differences were beyond the upper or lower 1% confidence intervals estimated from the permutation distribution. To identify loci that contain exposure-associated changes both at mRNA (expression data) and DNA level (CGH array), results from these two data analyses were combined. Six areas were common in the two analyses, namely 2p21-p16.3, 3p21.31, 5q35.2-q35.3, 16p13.3, 19p13.3.-13.1, and 22q12.3-q13.1 (Table 15). The data suggests that 2p21 could be simultaneously amplified in the exposed and deleted in the non-exposed patients' tumor samples, whereas 3p21.3-p21.1, 5q35.3, and 22q13.1 seem to be deleted among the tumors of the exposed group of patients. The largest significant region was detected on chromosome 19p13.3-19p13.1, showing a loss and down-regulation of genes in exposed patients and gain in the non-exposed patients.

Allelic imbalance on 19p13.3-13.1

Microsatellite (LOH) analysis was carried out to verify the exposure-associated changes on the p-arm of chromosome 19 and to reveal the extent of the aberration in 62 lung carcinomas from male patients that fell into three categories of exposure: heavy exposure, moderate occupational exposure, and no exposure to asbestos. 19 microsatellite markers spanning 22.3 Mbp region on 19p13.3-p13.1 were used for majority of the samples. For the ten paraffin samples, only five of the 19 markers producing fragments less than 200 bp were analyzed. 80% ( 20/25) of the exposed and 45% ( 13/29) of the non-exposed patients (p=0.0045) were found to be carriers of allelic imbalance (AI) on the 19p region in their tumor tissue. AI was also detected in 75% ( 6/8) of the moderately exposed patients. Allelic imbalance detected was in good accordance with the results indicated by the CGH array (Nymark et al., 2006).

Differences in AI frequencies were observed between histological tumor types. In the exposed groups, AI was prevalent regardless of histological type (Table 16). The results are presented for the combined group of heavily and moderately exposed patients because no obvious differences were detected in the AI frequencies between these two exposure groups. In the non-exposed group, AI was on the other hand detected commonly in adenocarcinomas. More thorough comparing between different lung cancer subtypes is, however, not possible due to limited group sizes.

The AI degree for individual markers ranged between 50-90% in exposed, 40-100% in moderately exposed, and 20-50% in non-exposed patients' tumor samples (only informative markers taken into account). When focusing into the differences in the frequency of AI as determined by individual markers, the frequency of chromosomal alterations was significantly higher with 11 out of 19 markers in the tumor samples from asbestos-exposed patients compared with the non-exposed patients. In most cases AI seemed to extend throughout the investigated 22 Mbp region, indicating a complete loss of the short arm of chromosome 19.

Additionally, 10% ( 3/29) of the non-exposed patients were found to have microsatellite instability (MSI) ranging throughout the region studied. When further assessing MSI with the colon MSI marker BAT-26, two of the three cases (54 and 60) showing high instability in the individual markers also showed instability in this marker. As MSI cases were detected among moderately exposed and non-exposed patients, MSI doesn't seem to be a major player in asbestos-related cancer and further analyses were not conducted.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications and human genomic data (e.g. GenBank accession numbers) cited herein are hereby incorporated by reference in their entirety for all purposes to the same extent as if each individual publication, patent, patent application, sequence data were specifically and individually indicated to be so incorporated by reference.

### Tables

TABLE 1
Patient samples

| Sample | | | Asbestos | Smoking | | | | |
|---|---|---|---|---|---|---|---|---|
| nr. | Sex | Age | fiber* | cig/day | PKY^{†} | Age-start | Age-stop | Diagnosis |

Exposed patients

| | | | | | | | | |
|---|---|---|---|---|---|---|---|---|
| 1 | M | 64 | 72.9 | 20 | 33 | 15 | 48 | AC |
| 2^{‡} | M | 59 | 12.6 | 50 | 105 | 17 | — | AC |
| 3^{‡} | M | 59 | 35.0 | 20 | 36 | 19 | 55 | AC |
| 4^{‡} | M | 65 | 9.4 | 10 | 25 | 16 | — | AC |
| 5^{‡} | M | 65 | 10.8 | 15 | 23 | 20 | 50 | AC |
| 6^{‡} | M | 63 | 10.8 | 17 | 27 | 16 | 60 | SCC |
| 7^{‡} | M | 62 | 6.0 | 30 | 65 | 14 | 57 | SCC |
| 8^{‡} | M | 65 | 5.9 | 20 | 32 | 18 | — | SCC |
| 9^{‡} | M | 57 | 6.6 | 20 | 36 | 17 | 53 | SCC |
| 10^{‡} | M | 67 | 8.4 | 20 | 20 | 36 | 56 | LCLC |
| 11^{‡} | M | 66 | 19.0 | 15 | 35 | 17 | 61 | LCLC |
| 12^{‡} | M | 58 | 90 | 30 | 65 | 15 | — | LCLC |
| 13 | M | 64 | 145 | 20 | 22 | 19 | 43 | AC/SCC |
| 14 | M | 62 | 12.8 | 23 | 55 | 14 | — | SCLC |
| mean | | 62.6 | 31.8 | 22.1 | 41.4 | 18.1 | | |

Non-exposed patients

| | | | | | | | | |
|---|---|---|---|---|---|---|---|---|
| 15^{‡} | M | 55 | 0.0 | 20 | 36 | 19 | — | AC |
| 16^{‡} | M | 69 | 0.0 | 20 | 47 | 23 | — | AC |
| 17 | M | 70 | 0.0 | 15 | 38 | 18 | 68 | AC |
| 18^{‡} | M | 65 | 0.0 | 20 | 52 | 12 | — | AC |
| 19 | M | 55 | 0.1 | 28 | 54 | 16 | 55 | AC |
| 20^{‡} | M | 67 | 0.0 | 25 | 50 | 27 | — | AC |
| 21 | M | 65 | 0.0 | 20 | 47 | 13 | 60 | SCC |
| 22 | M | 65 | 0.0 | 15 | 25 | 30 | 64 | SCC |
| 23^{‡} | M | 50 | 0.0 | 20 | 35 | 15 | — | SCC |
| 24 | M | 64 | 0.0 | 20 | 45 | 19 | — | SCC |
| 25^{‡,§} | M | 67 | 0.0 | 20 | 47 | 19 | 66 | SCC |
| 26^{‡} | M | 71 | 0.5 | 35 | 89 | 20 | — | LCLC |
| 27^{‡} | M | 72 | 0.5 | 22 | 36 | 18 | 49 | LCLC |
| 28^{‡} | M | 41 | 0.0 | 25 | 31 | 15 | 40 | AC/SCC |
| 29 | M | 64 | 0.0 | 30 | 66 | 17 | — | SCLC |
| mean | | 64.2 | 0.08 | 22.1 | 47.6 | 19 | | |

*million fibers/g dry lung tissue
^{†}pack years (20 cigarettes/day)
^{‡}used in array CGH
^{§}only used in array CGH
AC, adenocarcinoma;
SCC, squamous cell carcinoma;
LCLC, large cell lung cancer;
AC/SCC, adenosquamous cell carcinoma;
SCLC, small cell lung cancer

TABLE 2
Classical CGH results. Chromosomal gains and deletions in lung tumors from
14 asbestos-exposed and 14 matched non-exposed patients.

| Histol. | Sample | Gains and amplifications | Deletions | tot. |
|---|---|---|---|---|
| | Exposed | ≧1.17 | ≦0.85 | 78 |
| AC | 1 | normal karyotype | | 0 |
| | 2 | 1q21.2-q43, 2p22-p25.1, 6p, 8q12-q24, 8q24.2-qter, | | 7 |
| | | 12p11.2-pter, 16p11.2-p13.3, 17q24 | | |
| | 3 | 1q21.2-qter, 2p22-p23, 4p13-pter, 5p, 7q, | | 8 |
| | | 12q13.2-qter, 17q11.2-q25, 20q11.2-qter | | |
| | 4 | 17q12-q21.3 | | 1 |
| | 5 | 1q, 2p23-p24, 5p13.2-pter, 8q24.1-q24.3, 9q, | 9p21-pter, | 12 |
| | | 12p-q21.3, 15q24-26.1, 17q24-q25, 19q, 20q | 10 | |
| SCC | 6 | 8q23-q24.1 | | 1 |
| | 7 | 1q23-qter, 2p21-p25.1, 3q13.3-q29, 5p14-p15.3, | | 6 |
| | | 8q24.1-qter, 17q21.1-q25 | | |
| | 8 | 3q13.3-qter | | 2 |
| | 9 | 1q41-q42.1, 20q | 2 | |
| LCLC | 10 | 1q21.1-qter, 2p, 3q21-qter, 5p, 8q21.3-qter, | | 6 |
| | | 12p-q21.3 | | |
| | 11 | 2p21-p25.2, 2q14.1-q22, 2q36-37.3, 3q21-qter, | | 7 |
| | | 8q24.1-24.3, 15qcen-q22, 15q23-25, 15q26-qter, | | |
| | | 17q21.1-q25 | | |
| | 12 | 3p21.2-p22, 7q31.3-qter | | 2 |
| AC/SCC | 13 | 1q31-qter, 2p22-p24, 2q21.1-22, 7q22-q35, | | 5 |
| | | 8q24.1-q24.3 | | |
| SCLC | 14 | 1q21.3-qter, 2p, 2q14.1-q23, 2q35-q37.3, | 3p, 5q, 9p, | 19 |
| | | 3qcen-201, 3q21-q24, 3q25-qter, 5p13.3-15.1, | 10p, 14 | |
| | | 6pter-q22.3, 7q32-qter, 8, 11, 17q12-q25, 18, | | |
| | | 19p, 19q, 20p, 20q | | |
| | Non- | ≧1.17 | ≧0.85 | 77 |
| | exposed | | | |
| AC | 15 | 1q32.2-q42.1, 5p14-pter, 8q24.3-qter, 17q21.2-q25 | | 4 |
| | 16 | 11q23.1 | | 1 |
| | 17 | 7q32-qter | | 1 |
| | 18 | normal karyotype | | 0 |
| | 19 | 1q, 2q14.3-22, 11q23.3-24, 17q12-q25, 20 | | 6 |
| | 20 | 5p | | 1 |
| SCC | 21 | 3q25.3-q27, 17q12-q25 | | 2 |
| | 22 | 1q31-q42.3, 2p14-p24, 3q21-qter, 5q31.1-q34, 6p12-p21.3, | | 7 |
| | | 7, 8q22.1-qter, 17q21.3-q24 | | |
| | 23 | 3q22-qter, 5p14-pter, 7pcen-p21, 8q, 9p13-pcen, 9q, | 9p23-pter | 13 |
| | | 11q13.3-q22.1, 12p12.1-p13.3, 12q13.3-q21.3, | | |
| | | 17q21.3-q22, 20q, 22 | | |
| | 24 | 1qcen-q31, 2p-q22, 2q32.1-qter, 3qcen-26.1, 3q26.2-qter, | 3p, 4, 5qcen-q31.3, 7p, | 23 |
| | | 5p, 6p21.3-pter, 6q24-qter, 7q, 8q23-qter, 11, | 14qcen-q23, 15qcen-q21.1, | |
| | | 12p, 12q, 13, 15q23-qter, 17q, 19p-q11.3, 19q12-q13.2, | 18q21.3-qter | |
| | | 19q13.3-qter, 20, 22 | | |
| LCLC | 26 | 1q32.1-q42.2 | | 1 |
| | 27 | 12p12.1-p13.3 | | 1 |
| AC/SCC | 28 | 1q, 5q31.1-q35.1, 8q23-q24.3, 20q | | 4 |
| SCLC | 29 | 1p31.2-32.13, 1p32.2-p36.1, 1p36.2-pter, 1q, 5p, | 6qcen-23.1, 6q24-27, | 13 |
| | | 6p21.1-pter, 8, 17q12-q25, 18p-q12.1, 18q12.2-q21.2, | 10q22.1-qter | |
| | | 18q21.3-qter1, 20p, 20q, 21, 22 | | |

(Bold = high-level amplification;
Abbreviations:
AC, adenocarcinoma;
SCC, squamous cell carcinoma;
LCLC, large cell lung cancer;
AC/SCC, adeno-squamous cell carcinoma;
SCLC, small cell lung cancer.)

TABLE 3
Differing altered regions between the asbestos-exposed and non-exposed lung cancer patients achieved with array CGH.

| | Chromosomal | | Size | | | |
|---|---|---|---|---|---|---|
| Chromosomal | position (bp)* | | (Mbp) | Nr. | | |
| region | start | stop | UCSC | genes/region^{†} | Type of aberration | Fragile sites^{‡} |
| 1p36.12-p36.11 | 23500515 | 24426781 | 0.93 | 11/18 | AMP in exposed | FRA1A, fra(1)(p36) |
| 1q21.2 | 147049272 | 147599667 | 0.55 | 12/19 | (AMP in exp?) | FRA1F, fra(1)(q21) |
| 2p21-p16.3 | 45527471 | 48518085 | 2.99 | 12/14 | AMP in exposed | |
| 3p21.31 | 48530340 | 49429317 | 0.9 | 7/14 | DEL in exposed | |
| 4q31.21 | 145931414 | 147128135 | 1.2 | 6/7 | DEL in non-exposed | FRA4C, fra(4)(q31.1) |
| 5q35.2-q35.3 | 175775918 | 178511817 | 2.74 | 14/27 | DEL in exposed + AMP in non-exposed | FRA5G, fra(5)(q35) |
| 9q32 | 112313202 | 114440305 | 2.13 | 10/13 | DEL in exposed + AMP in non-exposed | FRA9E, fra(9)(q32) and FRA9B, |
| | | | | | | fra(9)(q32) |
| 9q33.3-q34.11 | 127249352 | 128990540 | 1.74 | 15/25 | DEL in exposed + (AMP in non- | |
| | | | | | exposed?) | |
| 9q34.13-q34.3 | 132796808 | 136547881 | 3.75 | 18/29 | DEL in exposed + AMP in non-exposed | |
| 11p15.5 | 780476 | 2547429 | 1.77 | 13/21 | DEL in exposed + AMP in non-exposed | |
| 11q12.3-q13.1 | 62517312 | 64095160 | 1.58 | 11/22 | AMP in non-exposed | FRA11H, fra(11)(q13) |
| 11q13.2 | 65886588 | 67191050 | 1.3 | 9/18 | AMP in non-exposed | FRA11A, fra(11)(q13) |
| 14q11.2 | 22004518 | 23616339 | 1.61 | 12/21 | AMP in non-exposed | |
| 16p13.3 | 258760 | 3399193 | 3.14 | 27/51 | AMP in non-exposed | |
| 17p13.3-p13.1 | 1194934 | 8156236 | 6.96 | 44/84 | DEL in exposed + AMP in non-exposed | |
| 19p13.3-p13.11 | 367882 | 18901114 | 18.53 | 133/233 | DEL in exposed (+AMP in non-exp?) | FRA19B, fra(19)(p13) |
| 22q12.3-q13.1 | 34861230 | 36292422 | 1.43 | 10/22 | (AMP in non-exp?) | FRA22A, fra(22)(q13) |
| Xq28 | 147672966 | 149603500 | 1.93 | 9/15 | AMP in exposed | FRAXE, fra(X)(q28) |

*base pair obtained by blasting array probe sequence in USCS Blat
^{†}number of genes within the region with different copy number between the exposed and non-exposed patients samples
^{‡}obtained from Entez, Gene
AMP, amplification;
DEL, deletion

TABLE 4
Cancer patient data

| | | HEAVY | NO EXPOSURE | NO EXPOSURE | MODERATE |
|---|---|---|---|---|---|
| | | EXPOSURE | array | LOH | EXPOSURE |
| | | n = 14 | n = 14 | n = 29 | n = 8 |
| Histology | AC | 5 | 6 | 12 | 4 |
| | SCC | 4 | 4 | 11 | 3 |
| | LCLC | 3 | 2 | 2 | 1 |
| | SCLC | 1 | 1 | 2 | — |
| | AC-SCC | 1 | 1 | 2 | — |
| asbestos¹ | (mean ± SD) | 31.8 ± 41.8 | 0.1 ± 0.2 | 0.1 ± 0.2 | 2.6 ± 1.0 |
| Age | (mean ± SD) | 62.6 ± 3.2 | 62.5 ± 9.0 | 62.0 ± 11.4 | 66.6 ± 9.9 |
| stage² | I | 7 | 6 | 11 | 3 |
| | II | 1 | 2 | 3 | 2 |
| | III | 3 | 3 | 7 | 3 |
| | IV | 2 | 2 | 4 | |
| grade² | I | 1 | — | — | — |
| | II | 3 | 3 | 9 | 4 |
| | III | 8 | 8 | 13 | 3 |
| smoking³ | non | — | — | — | 1 |
| | ex | 9 | 7 | 14 | 4 |
| | current | 5 | 7 | 15 | 2 |
| PY⁴ | (mean ± SD) | 41.4 ± 23.6 | 48.1 ± 15.0 | 41.1 ± 16.5 | 44.7 ± 26.0 |
| smok. years | (mean ± SD) | 35.8 ± 8.4 | 42.1 ± 8.0 | 40.5 ± 10.3 | 41.3 ± 15.0 |

¹mean fibers/g dried lung
²stage and grade missing for one non-exposed patient and grade for one intermediate exposed.
³ex smokers = stopped smoking more than 6 months prior to operation, smoking data missing for one intermediate exposed patient.
⁴PY = pack years

TABLE 5
The most significant 47 genes found in the correlation analysis separating the
lung carcinomas of 14 asbestos-exposed patients from the lung carcinomas of 14 non-
exposed patients

| Rank¹ | Probe Set ID² | Accession³ | Gene Symbol | Location⁴ | P-value⁵ | Correlation⁶ |
|---|---|---|---|---|---|---|
| 1 | 220127_s_at | NM_017703.1 | FBXL12 | 19p13.2 | 0.0001 | −0.90 |
| 2 | 210365_at | D43967.1 | RUNX1 | 21q22.3 | 0.0004 | −0.89 |
| 3 | 204594_s_at | NM_013298.1 | FLJ20232 | 22q13 | 0.0075 | −0.88 |
| 4 | 217147_s_at | AJ240085.1 | TRAT1 | 3q13 | 0.0395 | −0.87 |
| 5 | 208030_s_at | NM_001119.2 | ADD1 | 4p16.3 | 0.0004 | 0.86 |
| 6 | 217580_x_at | AW301806 | ARL6IP2 | 2p22.2-p22.1 | 0.0126 | −0.86 |
| 7 | 202801_at | NM_002730.1 | PRKACA | 19p13.1 | 0.2547 | −0.86 |
| 8 | 212517_at | AL132773 | ATRN | 20p13 | 0.0141 | 0.85 |
| 9 | 203241_at | NM_003369.1 | UVRAG | 11q13.5 | 0.0001 | −0.84 |
| 10 | 208994_s_at | AI638762 | PPIG | 2q31.1 | 0.0380 | −0.84 |
| 11 | 209494_s_at | AI807017 | ZNF278 | 22q12.2 | 0.0609 | −0.84 |
| 12 | 208442_s_at | NM_000051.1 | ATM | 11q22-q23 | 0.0012 | −0.84 |
| 13 | 221971_x_at | BE672818 | | 10q | 0.0179 | −0.84 |
| 14 | 221104_s_at | NM_018376.1 | NIPSNAP3B | 9q31.1 | 0.0822 | −0.84 |
| 15 | 213094_at | AL033377 | GPR126 | 6q24.1 | 0.0091 | 0.83 |
| 16 | 209471_s_at | L00634.1 | FNTA | 8p22-q11 | 0.0003 | −0.83 |
| 17 | 204834_at | NM_006682.1 | FGL2 | 7q11.23 | 0.0001 | −0.83 |
| 18 | 205884_at | NM_000885.2 | ITGA4 | 2q31.3 | 0.0445 | −0.83 |
| 19 | 205673_s_at | NM_024087.1 | ASB9 | Xp22.2 | 0.0110 | 0.83 |
| 20 | 204527_at | NM_000259.1 | MYO5A | 15q21 | 0.0273 | −0.83 |
| 21 | 210835_s_at | AF222711.1 | CTBP2 | 10q26.13 | 0.0861 | 0.83 |
| 22 | 207633_s_at | NM_005592.1 | MUSK | 9q31.3-q32 | 0.0956 | −0.82 |
| 23 | 210187_at | BC005147.1 | FKBP1A | 20p13 | 0.0295 | −0.82 |
| 24 | 219174_at | NM_025103.1 | CCDC2 | 9p21.2 | 0.0866 | 0.82 |
| 25 | 203892_at | NM_006103.1 | WFDC2 | 20q12-q13.2 | 0.0155 | 0.82 |
| 26 | 219613_s_at | NM_016539.1 | SIRT6 | 19p13.3 | 0.0592 | −0.82 |
| 27 | 203412_at | NM_006767.1 | LZTR1 | 22q11.1-q11.2 | 0.0014 | −0.82 |
| 28 | 219666_at | NM_022349.1 | MS4A6A | 11q12.1 | 0.0104 | −0.82 |
| 29 | 212215_at | AB007896.1 | PREPL | 2p22.1 | 0.0194 | 0.82 |
| 30 | 217718_s_at | NM_014052.1 | YWHAB | 20q13.1 | 0.0398 | 0.82 |
| 31 | 204288_s_at | NM_021069.1 | ARGBP2 | 4q35.1 | 0.0098 | 0.82 |
| 32 | 201186_at | NM_002337.1 | LRPAP1 | 4p16.3 | 0.0218 | 0.81 |
| 33 | 219795_at | NM_007231.1 | SLC6A14 | Xq23-q24 | 0.0031 | 0.81 |
| 34 | 207922_s_at | NM_005882.2 | MAEA | 4p16.3 | 0.0265 | 0.81 |
| 35 | 221471_at | AW173623 | TDE1 | 20q13.1-13.3 | 0.0236 | 0.81 |
| 36 | 210915_x_at | M15564.1 | TRBV19, TRBC1 | 7q34 | 0.0106 | −0.81 |
| 37 | 205876_at | NM_002310.2 | LIFR | 5p13-p12 | 0.1244 | 0.81 |
| 38 | 219777_at | NM_024711.1 | G1MAP6 | 7q36.1 | 0.0000 | −0.81 |
| 39 | 206978_at | NM_000647.2 | CCR2 | 3p21 | 0.0076 | −0.81 |
| 40 | 218559_s_at | NM_005461.1 | MAFB | 20q11.2-q13.1 | 0.0022 | −0.80 |
| 41 | 209276_s_at | AF162769.1 | GLRX | 5q14 | 0.0072 | −0.80 |
| 42 | 214080_x_at | AI815793 | PRKCSH | 19p13.2 | 0.0547 | −0.80 |
| 43 | 204523_at | NM_003440.1 | ZNF140 | 12q24.32-q24.33 | 0.0000 | 0.80 |
| 44 | 34221_at | D83778 | KIAA0194 | 5q33.1 | 0.0036 | −0.79 |
| 45 | 210895_s_at | L25259.1 | CD86 | 3q21 | 0.0686 | −0.79 |
| 46 | 33197_at | U39226 | MYO7A | 11q13.5 | 0.2430 | −0.79 |
| 47 | 205597_at | NM_025257.1 | C6orf29 | 6p21.3 | 0.1272 | 0.79 |

¹Genes ranked according to their correlation with exposure status (asbestos-exposed vs. non-exposed)
²Affymetrix probe ID
³The GenBank accession number
⁴The chromosomal location of the target DNA sequence
⁵P-value calculated for the differential expression of the gene in asbestos-exposed vs. non-exposed. Signals were scaled with respect to their matched normal lung signals or to the mean signal of the normal lung samples from the same exposure group.
⁶Correlation coefficient of gene expression with exposure status (asbestos-exposed vs. non-exposed). Signals of the asbestos-associated tumors were scaled by the median signal of the non-associated tumors and the signals of the non-associated tumors by the median signal of the asbestos-associated tumors.

TABLE 6
Combined results from gene expression
and DNA aberration profiling

| Chromosomal | Size | Bp position | | Non- |
|---|---|---|---|---|
| region | (Mbp) | (USCS) | Exposed | exposed |
| 2p21-p16.3 | 3.00 | 45527471-48530340 | Gain | Loss |
| 3p21.31 | 0.90 | 48530340-49429317 | Loss | |
| 5q35.2-q35.3 | 2.74 | 175775918-178511817 | Loss | |
| 16p13.3 | 3.14 | 258760-3399193 | Loss | |
| 19p13.3-p13.1 | 18.53 | 367882-18901114 | Loss | Gain |
| 22q12.3-q13.1 | 1.43 | 34861230-36292422 | Loss | |

TABLE 7
Fragment analysis results for LOH at 19p13.3-12 in lung carcinomas of 14 heavily asbestos-exposed,
8 moderately asbestos-exposed, and of 29 non-exposed patients.

| | Microsatellite marker¹ | | | | | | | | | | | | | | | | | | | |
|---|---|---|---|---|---|---|---|---|---|---|---|---|---|---|---|---|---|---|---|---|
| Case | | | | | | | | | | | | | | | | | | | | BAT |
| no. | 814 | 883 | 878 | 424 | 894 | 216 | 177 | 1034 | 873 | 884 | 916 | 583 | 535 | 906 | 221 | 840 | 917 | 895 | 568 | 26 |

Heavy exposure²

| | | | | | | | | | | | | | | | | | | | | |
|---|---|---|---|---|---|---|---|---|---|---|---|---|---|---|---|---|---|---|---|---|
| 20 | I³ | I | I | I | I | I | I | I | I | I | NI | I | I | I | I | I | I | NI | I | I |
| 23 | AI | NI | AI | AI | AI | AI | NI | AI | AI | AI | AI | AI | I | AI | AI | AI | NI | NI | NI | I |
| 45 | AI | AI | AI | NI | AI | AI | AI | NI | AI | AI | NI | AI | AI | AI | AI | NI | AI | NI | AI | I |
| 123 | AI | NI | NI | AI | NI | AI | AI | AI | AI | AI | AI | NI | NI | AI | NI | AI | NI | NI | NI | I |
| 155 | NI | I | I | I | I | AI | I | I | I | I | I | AI | I | I | I | AI | I | NA | I | I |
| 170 | AI | NI | NI | AI | AI | AI | NI | AI | NI | AI | NI | AI | AI | AI | AI | AI | I | I | NI | I |
| 185 | AI | I | I | AI | I | AI | I | NI | NI | AI | I | AI | I | AI | I | AI | NI | NA | NI | I |
| 188 | I | I | I | I | I | NI | I | NI | NI | I | I | I | AI | I | I | NI | NI | AI | I | I |
| 191 | AI | NI | AI | I | AI | AI | I | NI | NI | AI | AI | NI | I | I | AI | I | NA | NI | NA | I |
| 245 | NI | AI | AI | I | I | NI | I | AI | NI | AI | I | AI | AI | I | AI | AI | I | NA | AI | I |
| 252 | AI | AI | AI | AI | AI | AI | AI | NI | NI | AI | AI | AI | AI | AI | AI | NI | AI | NI | AI | I |
| 279 | AI | NA | AI | I | NI | AI | AI | AI | AI | AI | AI | AI | AI | AI | AI | AI | AI | AI | AI | I |
| 289 | AI | NI | I | AI | AI | AI | I | NI | AI | AI | AI | AI | I | AI | I | NI | I | NA | AI | I |
| 306 | AI | NI | AI | AI | NI | AI | AI | AI | AI | AI | AI | AI | AI | AI | AI | AI | AI | AI | AI | I |

Moderate exposure

| | | | | | | | | | | | | | | | | | | | | |
|---|---|---|---|---|---|---|---|---|---|---|---|---|---|---|---|---|---|---|---|---|
| 11 | NI | AI | NI | AI | NI | I | MSI | I | NI | I | I | MSI | AI | AI | I | NI | NA | NA | I | MSI |
| 78 | AI | NI | NI | NI | I | AI | NI | I | I | I | AI | AI | AI | AI | NI | NI | NI | NA | NI | I |
| 111 | AI | AI | AI | AI | AI | AI | AI | AI | AI | AI | AI | NI | I | I | AI | I | NA | NA | NA | I |
| 121 | NI | AI | AI | I | AI | AI | AI | AI | AI | AI | AI | AI | AI | AI | AI | AI | NA | AI | AI | I |
| 131 | AI | AI | NI | AI | AI | AI | AI | AI | NI | AI | AI | AI | I | I | AI | AI | I | NI | I | I |
| 143 | AI | NI | NI | NI | NI | AI | AI | AI | NI | AI | AI | NI | AI | NI | AI | NA | AI | MSI | AI | MSI |
| 189 | I | NI | NI | AI | NI | AI | AI | AI | AI | AI | I | AI | NI | AI | NA | NA | I | NI | AI | I |
| 260 | I | NI | NI | AI | NA | I | I | AI | I | I | NI | NA | I | I | NA | NA | NA | NA | NA | I |

No exposure

| | | | | | | | | | | | | | | | | | | | | |
|---|---|---|---|---|---|---|---|---|---|---|---|---|---|---|---|---|---|---|---|---|
| 13 | I | I | I | AI | I | I | NI | I | NA | I | I | I | I | I | I | I | I | NA | I | I |
| 14 | I | I | I | I | I | I | I | I | I | I | I | I | I | I | I | I | I | I | I | I |
| 22 | AI | NI | AI | AI | AI | NI | AI | AI | AI | AI | AI | AI | AI | AI | AI | NI | AI | AI | NI | I |
| 46 | NI | NI | AI | NI | NI | AI | I | AI | I | I | NI | AI | AI | I | I | NI | NI | NA | NI | I |
| 48 | AI | NI | NI | NI | NA | AI | NI | AI | AI | AI | AI | AI | AI | NI | NA | AI | I | NI | NI | I |
| 55 | I | I | I | I | I | NI | AI | I | I | NI | I | AI | AI | I | I | I | NI | NA | NI | I |
| 56 | AI | NI | AI | AI | AI | AI | AI | AI | AI | AI | NI | AI | AI | AI | AI | NI | NI | NI | AI | I |
| 57 | I | I | I | I | I | I | I | AI | I | I | I | I | I | I | I | I | I | I | NI | I |
| 62 | MSI | MSI | MSI | MSI | I | AI | MSI | I | MSI | MSI | MSI | NI | MSI | MSI | MSI | I | MSI | NA | MSI | MSI |
| 63 | I | I | NI | NI | I | I | NI | AI | I | I | I | I | I | I | NI | I | NI | AI | I | I |
| 80 | NI | I | I | NI | I | NI | I | I | NI | NI | I | AI | NI | NI | I | NI | I | NI | I | I |
| 99 | NI | AI | AI | AI | NA | AI | I | AI | AI | AI | NI | AI | AI | AI | AI | NI | NI | NA | AI | I |
| 107 | AI | NI | NI | AI | AI | NI | AI | NI | NI | AI | NI | I | I | I | I | NI | NA | NA | NA | I |
| 136 | I | NI | NI | NI | NI | AI | AI | NI | I | I | AI | AI | I | AI | I | NA | NI | NA | AI | I |
| 139 | AI | NI | NI | NA | NA | I | I | NI | AI | AI | AI | AI | I | AI | AI | AI | I | AI | NI | I |
| 149 | I | MSI | MSI | MSI | MSI | I | MSI | I | MSI | MSI | MSI | I | I | MSI | MSI | I | I | NA | I | MSI |
| 154 | I | NI | I | I | I | NI | I | I | NI | I | I | I | I | I | I | I | NI | NA | I | I |
| 169 | AI | AI | AI | AI | AI | AI | AI | AI | NI | NA | AI | AI | AI | NA | AI | NI | AI | AI | AI | I |
| 182 | NI | NI | NA | NA | NA | I | I | I | NI | I | I | I | I | MSI | I | AI | NA | NA | NA | I |
| 194 | AI | AI | NA | AI | NA | NI | AI | AI | AI | AI | AI | NI | AI | AI | AI | I | NA | NA | NA | I |
| 197 | I | I | I | I | I | I | I | I | I | I | I | I | I | I | I | I | I | NA | NI | I |
| 239 | I | I | I | I | I | I | I | I | I | I | I | I | I | I | I | I | I | NA | NI | I |
| 240 | MSI | MSI | MSI | I | MSI | MSI | I | I | I | MSI | MSI | MSI | MSI | MSI | I | I | MSI | NA | I | I |
| 243 | I | I | NI | NI | I | I | I | I | I | I | I | NI | I | I | I | I | I | NA | NI | I |
| 246 | I | I | I | I | I | I | I | I | I | I | I | I | I | I | I | I | I | NA | NI | I |
| 255 | AI | AI | AI | NI | AI | NI | AI | AI | AI | AI | I | AI | NA | NA | NA | AI | NA | NA | NI | I |
| 261 | NI | I | NI | AI | AI | AI | AI | AI | AI | AI | AI | AI | AI | AI | AI | AI | NA | NA | NI | I |
| 278 | I | I | I | I | I | I | I | I | I | I | I | I | I | I | I | I | NI | NA | I | I |
| 280 | AI | NI | I | I | NI | AI | I | I | AI | AI | I | NI | I | AI | I | NI | AI | NA | AI | I |

¹Microsatellite marker, markers used in the study without the prefix 19S.
²Exposure categories: heavy exposure, patients with more than 5 million fibers/g dry-weight lung tissue; moderate exposure, patients with 1-5 million fibers/g dry-weight lung tissue; no exposure, patients with no history of asbestos-exposure and less than 0.5 million fibers/g dry-weight.
³LOH results: I, informative marker without changes; NI, non-informative marker; AI, allelic imbalance; MSI, microsatellite instability; NA, no result. P-values for the occurrence of AI in lung carcinomas of all exposed vs. non-exposed patients for microsatellite markers from 814 to 568 are 0.004, 0.000, 0.090, 0.110, 0.090, 0.001, 0.240, 0.030, 0.090, 0.040, 0.001, 0.001, 0.240, 0.030, 0.010, 0.006, 0.090, not available for 895, and 0.050, respectively. P-values for the occurrence of AI in lung carcinomas of patients with heavy exposure vs. no exposure for microsatellite markers from 814 to 568 are 0.004, 0.008, 0.160, 0.490, 0.260, 0.005, 0.740, 0.038, 0.130, 0.050, 0.010, 0.005, 0.320, 0.017, 0.038, 0.001, 0.050, not available for 895, 0.080, respectively.

TABLE 8
Allelic imbalance on 9q31.3-q34.3

| | | | | | | | | | TC repeat |
|---|---|---|---|---|---|---|---|---|---|
| | D9S1675 | D9S1683 | D9S930 | D9S289 | D9S302 | D9S1776 | D9S170 | D9S1872 | 121021696-121021941 |
| | 9q31.3 | 9q31.3 | 9q32 | 9q32 | 9q32 | 9q33.1 | 9q33.1 | 9q33.1 | 9q33.1 |
| ADENOCARCINOMA | AI/all informative cases | | | | | | | | |
| exp cases | 1/4 | 1/3 | 6/7 | 4/6 | 3/7 | 2/5 | 0/3 | 1/3 | 3/6 |
| exp AI % | 25% | 33% | 86% | 67% | 43% | 40% | 0% | 33% | 50% |
| nonexp AI % | 40% | 50% | 56% | 75% | 70% | 0% | 50% | 50% | 17% |
| nonexp cases | 2/5 | 2/4 | 5/9 | 6/8 | 7/10 | 0/4 | 2/4 | 3/6 | 1/6 |
| OTHER SUBTYPES | AI/all informative cases | | | | | | | | |
| exp cases | 5/7 | 3/3 | 9/11 | 10/11 | 10/12 | 3/7 | 5/6 | 5/8 | 9/9 |
| exp AI % | 71% | 100% | 82% | 91% | 83% | 43% | 83% | 63% | 100% |
| nonexp AI % | 29% | 40% | 73% | 58% | 60% | 17% | 40% | 57% | 45% |
| nonexp cases | 2/7 | 2/5 | 8/11 | 7/12 | 9/15 | 1/6 | 2/5 | 4/7 | 5/11 |
| ALL HIST. TYPES | AI/all informative cases | | | | | | | | |
| exp cases | 6/11 | 4/6 | 15/18 | 14/17 | 13/19 | 5/12 | 5/9 | 6/12 | 12/15 |
| exp AI % | 55% | 67% | 83% | 82% | 68% | 42% | 56% | 50% | 80% |
| nonexp AI % | 33% | 44% | 65% | 65% | 64% | 10% | 44% | 54% | 35% |
| nonexp cases | 4/12 | 4/9 | 13/20 | 13/20 | 16/25 | 1/10 | 4/9 | 7/13 | 6/17 |

| | AC repeat | | | | | |
|---|---|---|---|---|---|---|
| | 121168476-121168710 | D9S195 | D9S1116 | D9S1831 | D9S1793 | D9S1838 |
| | 9q33.1 | 9q33.1 | 9q33.2 | 9q34.11 | 9q34.2 | 9q34.3 |
| ADENOCARCINOMA | AI/all informative cases | | | | | |
| exp cases | 2/3 | 5/8 | 3/6 | 3/5 | 1/2 | 5/5 |
| exp AI % | 67% | 63% | 50% | 60% | 50% | 100% |
| nonexp AI % | 60% | 22% | 75% | 25% | 63% | 55% |
| nonexp cases | 3/5 | 2/9 | 6/8 | 2/8 | 5/8 | 6/11 |
| OTHER SUBTYPES | AI/all informative cases | | | | | |
| exp cases | 6/7 | 7/11 | 9/12 | 7/11 | 8/8 | 6/11 |
| exp AI % | 86% | 64% | 75% | 64% | 100% | 55% |
| nonexp AI % | 50% | 38% | 36% | 56% | 60% | 46% |
| nonexp cases | 2/4 | 6/13 | 5/14 | 9/16 | 6/10 | 6/13 |
| ALL HIST. TYPES | AI/all informative cases | | | | | |
| exp cases | 8/10 | 12/19 | 12/18 | 10/16 | 9/10 | 11/16 |
| exp AI % | 80% | 63% | 67% | 63% | 90% | 69% |
| nonexp AI % | 56% | 36% | 50% | 46% | 61% | 50% |
| nonexp cases | 5/9 | 8/22 | 11/22 | 11/24 | 11/18 | 12/24 |

TABLE 9
FISH results on lung tumors with three BAC probes on 9q32 and 9q34.3.

| | BAC probe | | | BAC probe | | | BAC probe | | |
|---|---|---|---|---|---|---|---|---|---|
| | probe 1 | | | probe 2 | | | probe 3 | | |
| | RP11-10i9 | | | RP11-357D21 | | | RP11-100C15 | | |
| | 9q32 | | | 9q32 | | | 9q34.3 | | |
| | del | norm | amp | del | norm | amp | del | norm | amp |
| ADENOCARCINOMAS | | | | | | | | | |
| exp cases | 1/5 | 3/5 | 1/5 | 5/21 | 9/21 | 7/21 | 1/18 | 10/18 | 7/18 |
| exp % | 20% | 60% | 20% | 24% | 43% | 33% | 6% | 56% | 39% |
| nonexp % | 40% | 40% | 20% | 18% | 53% | 29% | 29% | 36% | 36% |
| nonexp cases | 2/5 | 2/5 | 1/5 | 3/17 | 9/17 | 5/17 | 4/14 | 5/14 | 5/14 |
| OTHER SUBTYPES | | | | | | | | | |
| exp cases | 5/14 | 4/14 | 5/14 | 9/23 | 5/23 | 9/23 | 7/24 | 7/24 | 10/24 |
| exp % | 36% | 29% | 36% | 39% | 22% | 39% | 29% | 29% | 42% |
| nonexp % | 14% | 57% | 29% | 25% | 38% | 38% | 9% | 64% | 27% |
| nonexp cases | 1/7 | 4/7 | 2/7 | 4/16 | 6/16 | 6/16 | 1/11 | 7/11 | 3/11 |
| ALL HIST. TYPES | | | | | | | | | |
| exp cases | 6/19 | 7/19 | 6/19 | 14/44 | 14/44 | 16/44 | 8/42 | 17/42 | 17/42 |
| exp % | 32% | 37% | 32% | 32% | 32% | 36% | 19% | 40% | 40% |
| nonexp % | 25% | 50% | 25% | 21% | 45% | 48% | 20% | 48% | 32% |
| nonexp cases | 3/12 | 6/12 | 3/12 | 7/33 | 15/33 | 11/33 | 5/25 | 12/25 | 8/25 |

TABLE 10
Combination of allelic imbalance in 19p and deletions or gains by
FISH on 9q (BAC probe RP11-375D21) in lung tumors of asbestos-
exposed and non-exposed individuals

| | 19pn&9qn¹ | 19pn&9qd/a | 19pAI&9qn | 19pAI&9qd/a | Total |
|---|---|---|---|---|---|
| Exposure: | N (%) | N (%) | N (%) | N (%) | N |
| Exposed | 0 (0) | 3 (15) | 4 (20) | 13 (65) | 20 |
| Non-exposed | 6 (32) | 4 (21) | 4 (21) | 5 (26) | 19 |

¹Combinations: 19pn&9qn, normal 19p and 9q; 19pn&9qd/a, normal 19p and deletion or gain in 9q; 19pAI&9qn, allelic imbalance in 19p and normal 9q; 19pAI&9qd/a, allelic imbalance in 19p and deletion or gain in 9q

TABLE 11
Allelic imbalance on 2p16-p21 in lung tumors of asbestos-exposed and non-exposed patients.

| | Marker: | | | | | | | | | | |
|---|---|---|---|---|---|---|---|---|---|---|---|
| | D2S2328 | D2S2259 | D2S119 | D2S2298 | D2S2240 | D2S2378 | D2S391 | D2S2739 | D2S2251 | D2S2153 | D2S378 |
| asbestos | 20% | 56% | 50% | 40% | 50% | 88% | 57% | 70% | 57% | 38% | 40% |
| exposed | | | | | | | | | | | |
| non-exposed | 13% | 33% | 14% | 38% | 36% | 45% | 43% | 33% | 25% | 38% | 10% |

TABLE 12
Allelic imbalance on 16p13.3 in lung carcinomas of
asbestos-exposed and non-exposed patients.

| | D16S3024 | D16S3070 | D16S3082 | D16S475 | D16S3027 | D16S3072 |
|---|---|---|---|---|---|---|
| | 16p13.3 | 16p13.3 | 16p13.3 | 16p13.3 | 16p13.3 | 16p13.3 |
| ADENOCARCINOMA | AI/all informative cases | | | | | |
| exp cases | 0/4 | 1/4 | 0/4 | 0/3 | 1/4 | 1/6 |
| exp AI % | 0% | 25% | 0% | 0% | 25% | 17% |
| nonexp AI % | 17% | 20% | 20% | 50% | 20% | 29% |
| nonexp cases | 1/6 | 1/5 | 1/4 | 2/4 | 1/5 | 2/7 |
| OTHER SUBTYPES | AI/all informative cases | | | | | |
| exp cases | 4/8 | 1/3 | 3/8 | 5/7 | 6/8 | 5/7 |
| exp AI % | 50% | 33% | 38% | 71% | 75% | 71% |
| nonexp AI % | 25% | 57% | 43% | 38% | 29% | 29% |
| nonexp cases | 2/8 | 4/7 | 3/7 | 3/8 | 2/7 | 2/7 |
| ALL HIST. TYPES | AI/all informative cases | | | | | |
| exp cases | 4/12 | 2/7 | 3/12 | 5/10 | 7/12 | 6/13 |
| exp AI % | 33% | 29% | 25% | 50% | 58% | 46% |
| nonexp AI % | 21% | 42% | 36% | 42% | 25% | 29% |
| nonexp cases | 3/14 | 5/12 | 4/11 | 5/12 | 3/12 | 4/14 |

TABLE 13
Allelic imbalance on 5q35.3.

| | D5S425 | D5S2069 | D5S2111 | D5S408 |
|---|---|---|---|---|
| | 5q35.1 | 5q35.2 | 5q35.2 | 5q35.3 |
| | AI/all informative cases | | | |
| ADENOCARCINOMA | | | | |
| exp cases | 2/3 | 0/1 | 3/5 | 2/3 |
| exp AI % | 67% | 0% | 60% | 67% |
| nonexp AI % | 83% | 80% | 50% | 40% |
| nonexp cases | 5/6 | 4/5 | 2/4 | 2/5 |
| OTHER SUBTYPES | | | | |
| exp cases | 4/5 | 4/5 | 5/5 | 5/8 |
| exp AI % | 80% | 80% | 100% | 63% |
| nonexp AI % | 60% | 40% | 100% | 71% |
| nonexp cases | 3/5 | 2/5 | 6/6 | 5/7 |
| ALL HIST. TYPES | | | | |
| exp cases | 6/8 | 4/6 | 8/10 | 7/11 |
| exp AI % | 75% | 67% | 80% | 64% |
| nonexp AI % | 73% | 60% | 80% | 58% |
| nonexp cases | 8/11 | 6/10 | 8/10 | 7/12 |

TABLE 14
Array CGH results on 5q35.3. The CGH ratio indicates
the mean ratio of all probes on the array within the 5q
region. Orange = <−0.2; indicates a possible deletion
of the region, green = >0.2; indicates a
possible amplification.

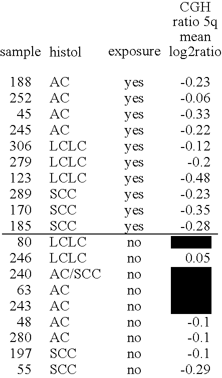

TABLE 15
Combined results from gene expression
and DNA aberration profiling

| Chromosomal | Size | | Asbestos- | Non- |
|---|---|---|---|---|
| region | (Mbp) | Bp position (USCS) | exposed | exposed |
| 2p21-p16.3 | 3.00 | 45527471-48530340 | Gain | Loss |
| 3p21.31 | 0.90 | 48530340-49429317 | Loss | No |
| | | | | aberration |
| 5q35.2-q35.3 | 2.74 | 175775918-178511817 | Loss | No |
| | | | | aberration |
| 16p13.3 | 3.14 | 258760-3399193 | No | Gain |
| | | | aberration | |
| 19p13.3-p13.1 | 18.53 | 367882-18901114 | Loss | Gain |
| 22q12.3-q13.1 | 1.43 | 34861230-36292422 | No | Gain |
| | | | aberration | |

TABLE 16
Prevalence of allelic imbalance on 19p in lung
carcinomas of asbestos-exposed and non-exposed
patients according to histological tumor type

| | ASBESTOS- | NON- | |
|---|---|---|---|
| | EXPOSED | EXPOSED | p-value^{II} |
| All histological tumor types | 26/33 (79%) | 13/29 (45%) | 0.008 |
| Adenocarcinomas^{I} | 9/13 (69%) | 8/12 (67%) | 1.0 |
| Other histological tumor types | 17/20 (85%) | 5/17 (29%) | 0.0004 |

^{I}The numbers of histological tumor types other than adenocarcinomas were not sufficient for separate statistical analysis on the relation of AI in 19p and asbestos-exposure
^{II}The permutation test (with 10 000 permutations) was used to detect differences in AI frequencies between the asbestos-exposed and non-exposed patients

TABLE 17
Characteristics of cancer patients and lung tumors
studied by expression array and array CGH

| | | HEAVILY | |
|---|---|---|---|
| | | ASBESTOS- | |
| | | EXPOSED | NON-EXPOSED |
| | | n = 14 | n = 14 |
| Gender | M/F | 14/— | 14/— |
| Age | mean ± SD | 62.6 ± 3.2 | 62.5 ± 9.0 |
| Asbestos^{I} | median | 11.7 (5.9-145) | 0.0 (0.0-0.5) |
| | (range) | | |
| Smoking^{II} | Non | — | — |
| | Ex | 9 | 7 |
| | Current | 5 | 7 |
| PY^{III} | mean ± SD | 41.4 ± 23.6 | 48.1 ± 15.0 |
| Smok. years | mean ± SD | 35.8 ± 8.4 | 42.1 ± 8.0 |
| Histology^{IV} | AC | 5 | 6 |
| | SCC | 4 | 4 |
| | LCLC | 3 | 2 |
| | SCLC | 1 | 1 |
| | AC-SCC | 1 | 1 |
| Stage^{V} | I | 7 | 6 |
| | II | 1 | 2 |
| | III | 3 | 3 |
| | IV | 2 | 2 |

^{I}Pulmonary asbestos fiber count in million per gram of dried lung
^{II}Ex-smokers had quitted smoking 6 months prior to operation or earlier. Smoking data is missing for one intermediately exposed patient.
^{III}PY, pack-years
^{IV}AC = adenocarcinoma, SCC = squamous cell carcinoma, LCLC = large cell carcinoma, SCLC = small cell carcinoma, AC-SCC = adenosquamous carcinoma
^{V}Stage is missing for one non-exposed patient.

TABLE 18
Characteristics of cancer patients and lung
tumors studied by microsatellite analysis

| | | | | MODER- |
|---|---|---|---|---|
| | | HEAVILY | | ATELY |
| | | ASBESTOS- | NON- | ASBESTOS- |
| | | EXPOSED | EXPOSED | EXPOSED |
| | | n = 25 | n = 29 | n = 8 |
| Gender | M/F | 25/— | 29/— | 8/— |
| Age | mean ± | 63.7 ± 6.2 | 62.5 ± 9.0 | 62.0 ± 11.4 |
| | SD | | | |
| Asbestos^{I} | median | 12.8 (5.9-8000) | 0.0 (0.0-0.50) | 2.3 (1.2-4.3) |
| | (range) | | | |
| Histology^{I} | AC | 9 | 12 | 4 |
| | SCC | 5 | 11 | 3 |
| | LCLC | 6 | 2 | 1 |
| | SCLC | 1 | 2 | — |
| | AC-SCC | 1 | 1 | — |
| | Giant | 1 | — | — |
| | cell carc. | | | |
| | Pleomorphic | 2 | 1 | — |
| | carc. | | | |

^{I}See Table 3 footnotes for definitions for histological tumor types and asbestos fiber count.

### REFERENCES

Björkqvist, A. M., Tammilehto, L., Nordling, S., Nurminen, M., Anttila, S., Mattson, K., and Knuutila, S. Comparison of DNA copy number changes in malignant mesothelioma, adenocarcinoma and large-cell anaplastic carcinoma of the lung. Br J Cancer, 77: 260-269, 1998.
Blyth K, Cameron E R, Neil J C. The RUNX genes: gain or loss of function in cancer. Nat Rev Cancer 2005; 5(5):376-87.
Bossolasco M, Lebel M, Lemieux N, Mes-Masson A M. The human TDE gene homologue: localization to 20q13.1-13.3 and variable expression in human tumor cell lines and tissue. Mol Carting 1999; 26(3):189-200.
Dano L, Guilly M M, M. Morlier, J P. Altmeyer, S. Vieth, P. El-Naggar, A K. Monchaux, G. Dutrillaux, B. Chevillard, S. CGH analysis of radon-induced rat lung tumors indicates similarities with human lung cancers. Genes Chromosomes Cancer 2000; 29(1):1-8.
De Rienzo A T, J R. Recent advances in the molecular analysis of human malignant mesothelioma. Clin Ter. 2000; 151(6):433-8.
Dopp, E. and Schiffmann, D. Analysis of chromosomal alterations induced by asbestos and ceramic fibers. Toxicol Lett, 96-97: 155-162, 1998.
el-Rifai, W., Larramendy, M., Bjorkqvist, A., Hemmer, S., and Knuutila, S. Optimization of comparative genomic hybridization using fluorochrome conjugated to dCTP and dUTP nucleotides. Lab Invest, 77: 699-700, 1997.
Fatma N, Jain A, Rahman Q. Frequency of sister chromatid exchange and chromosomal aberrations in asbestos cement workers. Br J Ind Med 1991; 48(2):103-5.
Finnis, M., Dayan, S., Hobson, L., Chenevix-Trench, G., Friend, K., Ried, K., Venter, D., Woollatt, E., Baker, E., and Richards, R. I. Common chromosomal fragile site FRA16D mutation in cancer cells, Hum Mol Genet, 14: 1341-1349, 2005.
Forozan, F., Karhu, R., Kononen, J., Kallioniemi, A., and Kallioniemi, O.-P. Genome screening by comparative genomic hybridization. Trends Genet, 13: 405-409, 1997.
Girard, L., Zochbauer-Muller, S., Virmani, A. K., Gazdar, A. F., and Minna, J. D. Genome-wide Allelotyping of Lung Cancer Identifies New Regions of Allelic Loss, Differences between Small Cell Lung Cancer and Non-Small Cell Lung Cancer, and Loci Clustering. Cancer Res, 60: 4894-4906, 2000.
Glover, T. Instability at chromosomal fragile sites. Recent Results Cancer Res., 154: 185-199, 1998.
Gupta N, Miyauchi S, Martindale R G, Herdman A V, Fodolsky R, Miyake K, et al. Upregulation of the amino acid transporter ATB0,+(SLC6A14) in colorectal cancer and metastasis in humans. Biochim Biophys Acta 2005; 1741(1-2):215-23.
Hellstrom I, Raycraft J, Hayden-Ledbetter M, Ledbetter J A, Sehummer M, McIntosh M, et al. The HE4 (WFDC2) protein is a biomarker for ovarian carcinoma. Cancer Res 2003; 63(13):3695-700.
Hoang J M, Cottu P H, Thuille B, Salmon R J, Thomas G, Hamelin R. BAT-26, an indicator of the replication error phenotype in colorectal cancers and cell lines. Cancer Res 1997; 57(2):300-3.
Hsieh, W. N.C., Hwang J J, Fang J S, Lin S P, Lin Y A, Huang T W, Chang W P. Evaluation of the frequencies of chromosomal aberrations in a population exposed to prolonged low dose-rate 60Co gamma-irradiation. Int J Radiat Biot, 78: 625-633, 2002.
Ionov Y, Nowak N, Perucho M, Markowitz S, Cowell J K. Manipulation of nonsense mediated decay identifies gene mutations in colon cancer Cells with microsatellite instability. Oncogene 2004; 23(3):639-45.
Jaurand M. Mechanisms of fiber-induced genotoxicity. Environ Health Perspectives 1997; 105(S5):1073-84.
Karjalainen A, Anttila S, Heikkilä L, Karhunen P, Vainio H. Asbestos exposure among Finnish lung cancer patients: Occupational history and fiber concentration in lung tissue. Am J Ind Med 1993; 23:461-471.
Karjalainen A, Anttila S, Vanhala E, Vainio H. Asbestos exposure and the risk of lung cancer in a general urban population. Scand J Work Environ Health 1994; 20(4):243-50.
Karjalainen A, Anttila S. Asbestos exposure and the risk of lung cancer in urban population. Houston, Tex.: Gulf Publishing Company; 1997.
Kettunen E, Anttila S, Seppanen J K, Karjalainen A, Edgren H, Lindstrom I, et al. Differentially expressed genes in nonsmall cell lung cancer: expression profiling of cancer-related genes in squamous cell lung cancer. Cancer Genet Cytogenet 2004; 149(2):98-106.
Larramendy, M., El-Rifai, W., and Knuutila, S. Comparison of fluorescein isothiocyanate- and Texas red-conjugated nucleotides for direct labeling in comparative genomic hybridization. Cytometry, 31: 174-179, 1998.
Leach, J. K., Van Tuyle, G., Lin, P.-S., Schmidt-Ullrich, R., and Mikkelsen, R. B. Ionizing Radiation-induced, Mitochondria-dependent Generation of Reactive Oxygen/Nitrogen. Cancer Res, 61: 3894-3901, 2001.
Li Q L, Kim H R, Kim W J, Choi J K, Lee Y H, Kim H M, et al. Transcriptional silencing of the RUNX3 gene by CpG hypermethylation is associated with lung cancer. Biochem Biophys Res Commun 2004; 314(1):223-8.
Lohani, M., Dopp, E., Becker, H.-H., Seth, K., Schiffmann, D., and Rahman, Q. Smoking enhances asbestos-induced genotoxicity, relative involvement of chromosome 1: a study using multicolor FISH with tandem labeling. Toxicol Lett, 136: 55-63, 2002.
Lounsbury K M, Stern M, Taatjes D, Jaken S, Mossman B T. Increased localization and substrate activation of protein kinase C delta in lung epithelial cells following exposure to asbestos. Am J Pathol 2002; 160(6):1991-2000.
Marczynski B, Czuppon A, Marek W, Reichel G, Baur X. Increased incidence of DNA double-strand breaks and anti-ds DNA antibodies in blood of workers occupationally exposed to asbestos. Human Experimental Toxicology 1994; 13(1).
Marczynski B, Rozynek P, Kraus T, Schlosser 5, Raithel H J, Baur X. Levels of 8-hydroxy-2′-deoxyguanosine in DNA of white blood cells from workers highly exposed to asbestos in Germany. Mutation Research/Genetic Toxicology and Environmental Mutagenesis 2000; 468(2):195-202.
Marsit C J, Hasegawa M, Hirao T, Kim D-H, Aldape K, Hinds P W, et al. Loss of Heterozygosity of Chromosome 3p21 Is Associated with Mutant TP53 and Better Patient Survival in Non-Small-Cell Lung Cancer. Cancer Res 2004; 64(23):8702-8707.
Medina P P, Carretero J, Ballestar E, Angulo B, Lopez-Rios F, Esteller M, et al. Transcriptional targets of the chromatin-remodelling factor SMARCA4/BRG1 in lung cancer cells. Hum Mol Genet. 2005; 14(7):973-82.
Michiels S, Koscielny S, Hill C. Prediction of cancer outcome with microarrays: a multiple random validation strategy. Lancet 2005; 365(9458):488-92.

Nelson H, Kelsey K. The molecular epidemiology of asbestos and tobacco in lung cancer. Oncogene 2002; 21(48):7284-8.

Nymark P, Wikman H, Ruosaari S, Hollmén J, Vanhala E, Karjalainen A et al. (2006). Identification of specific gene copy number changes in asbestos-related lung cancer. Cancer Res, 66, 5737-43.
Radak Z, Goto S, Nakamoto H, Udud K, Papai Z, Horvath I. Lung cancer in smoking patients inversely alters the activity of hOGG1 and hNTH1. Cancer Lett 2005; 219(2):191-5.
Reisman D N, Sciarrotta J, Wang W, Funkhouser W K, Weissman B E. Loss of BRG1/BRM in human lung cancer cell lines and primary lung cancers: correlation with poor prognosis. Cancer Res 2003; 63(3):560-6.
Ruosaari, S, and Hollmén, J. Image analysis for detecting faulty spots from microarray images. Lecture Notes In Computer Science, 2534: 259-266, 2002.
Safar A M, Spencer H, 3rd, Su X, Coffey M, Cooney C A, Ratnasinghe L D, et al. Methylation profiling of archived non-small cell lung cancer: a promising prognostic system. Clin Cancer Res 2005; 11(12):4400-5.
Sakakura C, Hagiwara A, Miyagawa K, Nakashima S, Yoshikawa T, Kin S, et al. Frequent downregulation of the runt domain transcription factors RUNX1, RUNX3 and their cofactor CBFB in gastric cancer. Int J Cancer 2005; 113(2):221-8.
Sanchez-Cespedes M, Ahrendt S A, Piantadosi S, Rosell R, Monzo M, Wu L, et al. Chromosomal Alterations in Lung Adenocarcinoma from Smokers and Nonsmokers. Cancer Res 2001; 61 (4): 1309-1313.
Sanchez-Cespedes M, Parrella P, Esteller M, Nomoto S, Trink B, Engles J M, et al. Inactivation of LKB1/STK11 is a common event in adenocarcinomas of the lung. Cancer Res 2002; 62(13):3659-62.
Selikoff I, Hammond E, Churg J. Asbestos exposure, smoking, and neoplasia. JAMA 1968; 204(2):106-12.
Shukla, A., Flanders, T., Lounsbury, K. M., and Mossman, B. T. The {gamma}-Glutamylcysteine Synthetase and Glutathione Regulate Asbestos-induced Expression of Activator Protein-1 Family Members and Activity. Cancer Res, 64: 7780-7786, 2004.
Suzuki, K., Ogura, T., Yokose, T., Nagai, K., Mukai, K., Kodama, T., Nishiwaki, Y., and Esumi, H. Loss of heterozygosity in the tuberous sclerosis gene associated regions in adenocarcinoma of the lung accompanied by multiple atypical adenomatous hyperplasia. Int J Cancer, 79: 384-389, 1998.
Takamochi K, Ogura T, Yokose T, Ochiai A, Nagai K, Nishiwaki Y, et al. Molecular analysis of the TSC1 gene in adenocarcinoma of the lung. Lung Cancer 2004; 46(3):271-281.
Upadhyay D, Kamp D W. Asbestos-Induced Pulmonary Toxicity: Role of DNA Damage and Apoptosis. Experimental Biology and Medicine 2003; 228(6):650-659.

Vainio H, Boffetta P. Mechanisms of the combined effect of asbestos and smoking in the etiology of lung cancer. Scandinavian Journal of Work, Environment & Health 1994; 20(4):235-42.
van't Veer L J, Dai H, van de Vijver M J, He Y D, Hart A A, Mao M, et al. Gene expression profiling predicts clinical outcome of breast cancer. Nature 2002; 415(6871):530-6.
Wikman H, Kettunen E, Seppanen J K, Karjalainen A, Hollmen J, Anttila S, et al. Identification of differentially expressed genes in pulmonary adenocarcinoma by using cDNA array. Oncogene 2002; 21(37):5804-13.
Wikman, H., Nymark, P., Väyrynen, A., Jarmalaite, S., Kallioniemi, A., Salmenkivi, K., Vainio-Siukola, K., Husgafvel-Pursiainen, K., Knuutila, S., Wolf, M., and Anttila, S. CDK4 Is a Probable Target Gene in a Novel Amplicon on 12q13.3-q14.1 in Lung Cancer. Genes Chromosomes Cancer. 42: 193-199, 2005.
Zainabadi, K., Benyamini, P., Chakrabarti, R., Veena, M. S., Chandrasekharappa, S. C., Gatti, R. A., and Srivatsan, E. S. A 700-kb physical and transcription map of the cervical cancer tumor suppressor gene locus on chromosome 11q13. Genomics, 85: 704-714, 2005.

1. A method of identifying lung cancers associated with asbestos-exposure, the method comprising steps of providing a sample of lung cancer cells taken from an individual suffering from lung cancer and detecting allelic imbalance (AI) in at least one of the following chromosomal regions of the lung cancer cells:
a) 19p13.3-p13.1;
b) 9q32-34.3;
c) 2p21-p16.3;
d) 16p13.3;
e) 22q12.3-q13.1; and
f) 5q35.3

2. The method according to claim 1, wherein the presence of AI in at least one of said regions indicates that the malignancy of the lung cancer cell is related to asbestos-exposure.

3. The method according to claim 1, wherein the chromosomal region is 19p13.3-p13.1.

4. The method according to claim 1, wherein the chromosomal region is 9q33.1.

5. The method according to claim 4, wherein the presence of AI in chromosomal region 19p13.3-p13.1 is assessed by the use of at least one of the following microsatellite markers: 19s814, 19S883, 19S878, 19S424, 19S894, 19S216, 19S177, 19S1034, 19S873, 19S884, 19S916, 19S583, 19S535, 19S906, 19S221, 19S840, 19S917, 19S895, and 19S568.

6. The method according to claim 1, wherein the presence of AI is determined by loss of heterozygosity (LOH) analysis.

7. The method according to claim 1, wherein the presence of AI is determined by preparing a gene expression profile.

8. The method according to claim 7, wherein the gene expression profile comprises expression data of at least one of the genes listed in Table 5.

9. The method according to claim 1, wherein the presence of AI is determined by the use of fluorescence in situ hybridization (FISH) technology.

10. The method according to claim 1, wherein the presence of AI is determined by the use of laser microdissection technology.

11. A kit comprising means for carrying out the method of claim 1.

12. The kit according to claim 11 for determining AI in at least one of the following chromosomal regions of a lung cancer cell:
a) 19p13.3-p13.1;
b) 9q32-34.3;
e) 2p21-p16.3;
d) 16p13.3;
e) 22q12.3-q13.1; and
f) 5q35.3.

13. A method of identifying a risk of lung cancer, the method comprising steps of providing a biological sample taken from an individual and detecting allelic imbalance (AI) in at least one of the following chromosomal regions of the lung cancer cells:
a) 19p13.3-p13.1;
b) 9q32-34.3;
c) 2p21-p16.3;
d) 16p13.3;
e) 22q12.3-q13.1; and
f) 5q35.3;
wherein the presence of AI in any of said chromosomal regions indicates altered risk of lung cancer.

14. The method according to claim 13, wherein said altered risk is elevated risk of lung cancer.

15. The method according to claim 13, wherein said biological sample is a sputum, bronchial washing, bronchoalveolar lavage, whole blood, plasma, or serum sample obtained from said individual.
